(19) United States
(12) Patent Application Publication
(10) Pub. No.: US 2013/0211344 A1
(43) Pub. Date: Aug. 15, 2013

(54) MEDICAL COMPONENTS HAVING COATED SURFACES EXHIBITING LOW FRICTION AND/OR LOW GAS/LIQUID PERMEABILITY

(71) Applicants: Nestor Rodriguez, Hamburg, NJ (US); Paolo Mangiagalli, Fontanil (FR)

(72) Inventors: Nestor Rodriguez, Hamburg, NJ (US); Paolo Mangiagalli, Fontanil (FR)

(21) Appl. No.: 13/519,553

(22) PCT Filed: Dec. 31, 2009
(86) PCT No.: PCT/IB09/56065

§ 371 Date: Jan. 10, 2013

(51) Int. Cl. A61M 5/315; B05D 5/00

(52) U.S. Cl. CPC A61M 5/31513; B05D 5/00
USPC 604/230; 427/2.3

(57) ABSTRACT

This invention relates to components useful for medical articles, such as a syringe assemblies, having sliding contact surface (s) coated with at least one coating layer, wherein the contact surface has an average surface roughness (Ra) ranging from about 10 nm to about 1700 nm and/or the coating layer has crystalline domains, the mass of the crystalline domains being at least about 20% of the total mass of the coating layer, and methods of making the same.

### BACKGROUND OF THE INVENTION

1. Field of the Invention

This invention relates to medical components, such as a syringe, tube or medical collection device, having coated surfaces exhibiting low friction and/or low gas/liquid permeability.

2. Description of Related Technology

Traditionally, containers for chemically sensitive materials have been made from inorganic materials such as glass. Glass containers offer the advantage that they are substantially impenetrable by atmospheric gases and thus provide a product with a long shelf life. However, glass containers can be fragile and expensive to manufacture.

More recently, lighter and less expensive containers made of polymeric materials are being used in applications in which traditional glass containers were used. These polymeric containers are less susceptible to breakage, lighter, and less expensive to ship than glass containers. However, polymeric containers can be permeable to gases, permitting atmospheric gases to pass through the polymeric container to the packaged product and also permitting gases in the packaged product to escape through the polymeric container, both of which undesirably degrade the quality and shelf life of the packaged product.

Whether the container is formed from glass or polymeric material, reactivity of the interior surface of the container with the contents of the container, such as biological materials and/or drugs, can be problematic. Trace components of the glass or polymeric material may migrate into the container contents, and/or components of the container contents may migrate or react with the interior surface of the container.

Also, certain devices, such as syringe barrels, require slow and controlled initiation and maintenance of sliding movement of one surface over another surface. It is well known that two stationary surfaces having a sliding relationship often exhibit sufficient resistance to initiation of movement that gradually increased force applied to one of the surfaces does not cause movement until a threshold force is reached, at which point a sudden sliding or shearing separation of the surfaces takes place. This sudden separation of stationary surfaces into a sliding relationship is herein referred to as “breakout” or “breakloose”.

“Breakout force” refers to the force required to overcome static friction between surfaces of a syringe assembly that has been previously moved in a sliding relationship, but has been stationary (“parked” or not moved) for a short period of time (for example, milliseconds to hours). A less well known but important frictional force is “breakloose force”, which refers to the force required to overcome static friction between surfaces of a syringe assembly that have not been previously moved in a sliding relationship or have been stationary for longer periods of time, often with chemical or material bonding or deformation of the surfaces due to age, sterilization, temperature cycling, or other processing.

Breakout and breakloose forces are particularly troublesome in liquid dispensing devices, such as syringes, used to deliver small, accurately measured quantities of a liquid by smooth incremental line to line advancement of one surface over a second surface. The problem also is encountered in devices using stopcocks, such as burets, pipets, addition funnels, and the like where careful dropwise control of flow is desired.

The problems of excessive breakout and breakloose forces are related to friction. Friction is generally defined as the resisting force that arises when a surface of one substance slides, or tends to slide, over an adjoining surface of itself or another substance. Between surfaces of solids in contact, there may be two kinds of friction: (1) the resistance opposing the force required to start to move one surface over another, conventionally known as static friction, and (2) the resistance opposing the force required to move one surface over another at a variable, fixed, or predetermined speed, conventionally known as kinetic friction.

The force required to overcome static friction and induce breakout or breakloose is referred to as the “breakout force” or “breakloose force”, respectively, and the force required to maintain steady slide of one surface over another after breakout or breakloose is referred to as the “sustaining force”. Three main factors, sticktion, inertia, and dimensional interference (including morphology) between the two surfaces contribute to static friction and thus to the breakout or breakloose force. The term “stick” or “sticktion” as used herein denotes the tendency of two surfaces in stationary contact to develop a degree of adherence to each other. The term “inertia” is conventionally defined as the indisposition to motion which must be overcome to set a mass in motion. In the context of the present invention, inertia is understood to denote that component of the breakout or breakloose force which does not involve adherence.

Breakout or breakloose forces, in particular the degree of sticktion, vary according to the composition and dimensional interference (related to morphology) of the surfaces. In general, materials having elasticity show greater sticktion than non-elastic materials. The length of time that surfaces have been in stationary contact with each other also influences breakout and/or breakloose forces. In the syringe art, the term “parking” denotes storage time, shelf time, or the interval between filling and discharge. Parking time generally increases breakout or breakloose force, particularly if the syringe has been refrigerated or heated during parking.

A conventional approach to overcoming breakout or breakloose has been application of a lubricant to a surface interface. Common lubricants used are silicone or hydrocarbon oils, such as mineral oils, peanut oil, vegetable oils, and the like. Such products have the disadvantage of being soluble in a variety of fluids, such as vehicles commonly used to dispense medicaments. In addition, hydrocarbon oil lubricants are subject to air oxidation resulting in viscosity changes and objectionable color development. Further, they are particularly likely to migrate from the surface to surface interface. Such lubricant migration is generally thought to be responsible for the increase in breakout or breakloose force with time in parking. As a separate issue, the lubricant can also migrate into the contained solution causing undesirable interactions with the active pharmaceutical ingredients or excipients.

Thus, there is a need for a lubricity mechanism to overcome high breakout and breakloose forces whereby smooth transition of two surfaces from stationary contact into sliding contact can be achieved. Also, there is a need for an improved barrier coating to prevent leaching of materials from a container or seal surface into the container contents and/or from the container contents into the container or seal surface, and to prevent gas and/or water permeability in medical articles, such as syringes, tubes and medical collection devices.

### SUMMARY OF THE INVENTION

In some non-limiting embodiments, the present invention provides an article of manufacture comprising a first component having a contact surface in frictional engagement with a contact surface of a second component, wherein at least one of the first component and the second component comprises a substrate having at least one coating layer on at least a portion of a surface of the substrate, wherein the contact surface of the first and/or second component has an average surface roughness (Rₐ) ranging from about 10 nm to about 1700 nm.

In some non-limiting embodiments, the present invention provides a medical article comprising a chamber, the chamber comprising a substrate having at least one coating layer on at least a portion of a surface of the substrate, wherein an outer surface of the chamber has an average surface roughness (Rₐ) ranging from about 10 nm to about 1700 nm.

In some non-limiting embodiments, the present invention provides a chamber for a medical article, the chamber having a contact surface adapted to sealingly engage a contact surface of a sealing member for a medical article, wherein the chamber comprises a substrate having at least one coating layer on at least a portion of a surface of the substrate, wherein the contact surface of the chamber has an average surface roughness (Rₐ) ranging from about 10 nm to about 1700 nm.

In some non-limiting embodiments, the present invention provides a sealing member for a medical article, the sealing member having a contact surface in sliding engagement with a contact surface of a chamber of a medical article and adapted to sealingly engage the contact surface of the chamber, wherein the sealing member comprises a substrate having at least one coating layer on at least a portion of a surface of the substrate, wherein the contact surface of the sealing member has an average surface roughness (Rₐ) ranging from about 10 nm to about 1700 nm.

In some non-limiting embodiments, the present invention provides an article of manufacture comprising a first component having a contact surface in frictional engagement with a contact surface of a second component, wherein at least one of the first component and the second component comprises a substrate having at least one coating layer on at least a portion of a surface of the substrate, the coating layer comprising crystalline domains, wherein the mass of the crystalline domains comprises at least about 20% of the total mass of the coating layer.

In some non-limiting embodiments, the present invention provides a medical article comprising a chamber, the chamber comprising a substrate having at least one coating layer on at least a portion of a surface of the substrate, the coating layer comprising crystalline domains, wherein the mass of the crystalline domains comprises at least about 20% of the total mass of the coating layer.

In some non-limiting embodiments, the present invention provides a chamber for a medical article, the chamber having a contact surface adapted to sealingly engage a contact surface of a sealing member for a medical article, wherein the chamber comprises a substrate having at least one coating layer on at least a portion of a surface of the substrate, the coating layer comprising crystalline domains, wherein the mass of the crystalline domains comprises at least about 20% of the total mass of the coating layer.

In some non-limiting embodiments, the present invention provides a sealing member for a medical article, the sealing member having a contact surface in sliding engagement with a contact surface of a chamber of a medical article and adapted to sealingly engage the contact surface of the chamber, wherein the sealing member comprises a substrate having at least one coating layer on at least a portion of a surface of the substrate, the coating layer comprising crystalline domains, wherein the mass of the crystalline domains comprises at least about 20% of the total mass of the coating layer.

Methods of inhibiting sticktion and friction between adjacent surfaces also are provided.

### BRIEF DESCRIPTION OF THE DRAWINGS

The foregoing summary, as well as the following detailed description, will be better understood when read in conjunction with the appended drawings. In the drawings:

FIG. 1 is an Fourier Transform Infrared Spectroscopy (FTIR) analysis of a semi-crystalline polytetrafluoroethylene-coated cyclic polyolefin substrate, according to the present invention;

FIG. 2 is an FTIR analysis of an Omniflex® fluoro-coated rubber stopper;

FIG. 3 is a Scanning Electron Microscopy (SEM) analysis of the semi-crystalline polytetrafluoroethylene-coated cyclic polyolefin substrate of FIG. 1, according to the present invention;

FIG. 4 is an SEM analysis of the Omniflex® fluoro-coated rubber stopper;

FIG. 5 is an SEM analysis of a semi-crystalline polytetrafluoroethylene-coated butyl rubber substrate according to the present invention;

FIG. 6 is an SEM analysis of a semi-crystalline polytetrafluoroethylene-coated silicon wafer substrate, according to the present invention;

FIG. 7 is an SEM analysis of a semi-crystalline polytetrafluoroethylene-coated butyl rubber stopper substrate, according to the present invention;

FIG. 8 is an SEM analysis of a semi-crystalline polytetrafluoroethylene-coated butyl rubber plate, according to the present invention;

FIG. 9 is an SEM analysis of a semi-crystalline polytetrafluoroethylene-coated glass substrate, according to the present invention;

FIG. 10 is an SEM analysis of a semi-crystalline polytetrafluoroethylene-coated fragment of a glass syringe barrel, according to the present invention;

FIG. 11 is an SEM analysis of a semi-crystalline polytetrafluoroethylene-coated fragment of a cyclic polyolefin syringe barrel, according to the present invention;

FIG. 12 is a photograph of a semi-crystalline polytetrafluoroethylene-coated butyl rubber stopper within a syringe barrel, according to the present invention;

FIG. 13 is a photograph of a semi-crystalline polytetrafluoroethylene-coated butyl rubber stopper within a syringe barrel, according to the present invention (left and right side) and a conventional butyl rubber siliconized stopper (center);

FIG. 14 is an X-ray diffraction (XRD) analysis of a semi-crystalline polytetrafluoroethylene-coated cyclic polyolefin substrate, according to the present invention;

FIG. 15 is an XRD analysis of an Omniflex® fluoro-coated rubber stopper;

FIG. 16 is an X-ray photoelectron spectroscopy (XPS) analysis of an Omniflex® fluoro-coated rubber stopper;

FIG. 17 is an X-ray photoelectron spectroscopy (XPS) analysis of a semi-crystalline polytetrafluoroethylene-coated butyl rubber stopper, according to the present invention;

FIG. 18 is an optical profilometry analysis of a semi-crystalline polytetrafluoroethylene-coated butyl rubber stopper, according to the present invention;

FIG. 19 is an optical profilometry analysis of a semi-crystalline polytetrafluoroethylene-coated butyl rubber stopper, according to the present invention;

FIG. 20 is an optical profilometry analysis of an Omniflex® fluoro-coated rubber stopper;

FIG. 21 is a graph of actuation and gliding force between semi-crystalline polytetrafluoroethylene-coated butyl rubber stoppers and non-lubricated glass barrels for four samples, according to the present invention;

FIG. 22 is a graph of infusion pump actuation force test results for a semi-crystalline polytetrafluoroethylene-coated rubber stopper and a conventional silicone oil lubricated barrel syringe assembly at a feed rate of 0.1 ml/hr, according to the present invention; and

FIG. 23 is a graph of infusion pump actuation force test results for an Omniflex® fluoro-coated rubber stopper and a conventional silicone oil lubricated barrel syringe assembly at a feed rate of 0.1 ml/hr.

### DETAILED DESCRIPTION

Other than in the operating examples, or where otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical values, however, inherently contain certain errors necessarily resulting from the standard deviation found in their respective testing measurements. Furthermore, when numerical ranges of varying scope are set forth herein, it is contemplated that any combination of these values inclusive of the recited values may be used.

Also, it should be understood that any numerical range recited herein is intended to include all sub-ranges subsumed therein. For example, a range of “1 to 10” is intended to include all sub-ranges between and including the recited minimum value of 1 and the recited maximum value of 10, that is, having a minimum value equal to or greater than 1 and a maximum value of equal to or less than 10. A range of “less than 5” includes all subranges below 5.

In some non-limiting embodiments, the present invention encompasses an article of manufacture comprising a first component having a contact surface in frictional engagement with a contact surface of a second component. The first component and/or the second component comprise a substrate having at least one coating layer on at least a portion of a surface of the substrate. The coated surface of the component is adapted to sealingly engage an adjoining surface of another component of the medical article. For example, the component can comprise a chamber or barrel having an inner surface adapted to sealingly engage an exterior surface of a sealing member for a medical article.

In some non-limiting embodiments, the contact surface has an average surface roughness (Rₐ) ranging from about 10 nm to about 1700 nm. While not intending to be bound by any theory, it is believed that roughness of the contact surface combined with surface energy and structural characteristics of the coating can provide cavities or a trapping layer for trapping a fluid (gas and/or liquid) which can improve gliding between contacting surfaces. The surface roughness can be adjusted to affect the gliding performance by: providing morphology favorable to trap fluid at the interface, without creating channels comprising overall container closure integrity, and adjusting the interface micro-contact distances to reduce structural ageing of gliding interfaces.

In other non-limiting embodiments, the coating layer comprises crystalline domains, wherein the mass of the crystalline domains comprises at least about 20% of the total mass of the coating layer. While not intending to be bound by any theory, it is believed that crystalline domains comprising generally lamellar crystalline particles provide planes which are capable of sliding relative to each other with reduced friction compared to surrounding amorphous domains. The lateral displacement can also be enhanced by the flexibility of the matrix. Also, a coating layer having both crystalline and amorphous domains can reduce permeability to gases.

Adjusting the crystallinity and roughness of the coating can impact the static and dynamic coefficient of friction, and can induce huge force variations in displacement of mating surfaces at slow speed (“stick-slip effect”).

In some non-limiting embodiments, the present invention encompasses a medical article comprising a chamber. The chamber comprises a substrate having at least one coating layer on at least a portion of a surface of the substrate. In some non-limiting embodiments, an outer surface of the chamber has an average surface roughness (Rₐ) ranging from about 10 nm to about 1700 nm. In other non-limiting embodiments, the coating layer comprises crystalline domains, wherein the mass of the crystalline domains comprises at least about 20% of the total mass of the coating layer.

In some non-limiting embodiments, the present invention encompasses a chamber for a medical article, the chamber having a contact surface adapted to sealingly engage a contact surface of a sealing member for a medical article. The chamber comprises a substrate having at least one coating layer on at least a portion of a surface of the substrate. In some non-limiting embodiments, the contact surface of the chamber has an average surface roughness (Rₐ) ranging from about 10 nm to about 1700 nm. In other non-limiting embodiments, the coating layer comprises crystalline domains, wherein the mass of the crystalline domains comprises at least about 20% of the total mass of the coating layer.

In some non-limiting embodiments, the present invention encompasses a sealing member for a medical article, the sealing member having a contact surface in sliding engagement with a contact surface of a chamber of a medical article and adapted to sealingly engage the contact surface of the chamber. The sealing member comprises a substrate having at least one coating layer on at least a portion of a surface of the substrate. In some non-limiting embodiments, the contact surface of the sealing member has an average surface roughness (Rₐ) ranging from about 10 nm to about 1700 nm. In other non-limiting embodiments, the coating layer comprises crystalline domains, wherein the mass of the crystalline domains comprises at least about 20% of the total mass of the coating layer.

In some non-limiting embodiments, either the first component or the second component have an average surface roughness (Rₐ) ranging from about 10 nm to about 1700 nm and/or a coating layer comprising crystalline domains, wherein the mass of the crystalline domains comprises at least about 20% of the total mass of the coating layer, the other component being uncoated or having a conventional coating such as silicone or other oil. In other non-limiting embodiments, both the first component and the second component have an average surface roughness (Rₐ) ranging from about 10 nm to about 1700 nm and/or a coating layer comprising crystalline domains, wherein the mass of the crystalline domains comprises at least about 20% of the total mass of the coating layer.

The respective contact surfaces of the chamber and the sealing member can be in frictional engagement. When used in a medical article, the effects of the present invention can reduce the force required to achieve breakout, breakloose, and/or sustaining forces, whereby transition of surfaces from stationary contact to sliding contact occurs without a sudden surge. When breakout or breakloose is complete and the surfaces are in sliding contact, they slide smoothly upon application of very low sustaining force. The effect achieved by the methods of the present invention can be of long duration, and articles, such as syringes, can retain the advantages of low breakout, low breakloose, and low sustaining forces for several years. When the chamber is part of a liquid dispensing device, small highly accurate increments of liquid may be dispensed repeatedly without sudden surges. Thus, a syringe including a chamber and/or sealing member treated according to the present invention can be used to administer a medicament to a patient without the danger of surges whereby accurate control of dosage and greatly enhanced patient safety are realized.

Another useful feature of the present invention is that the coating can function as a barrier to reduce permeability to oxygen and other gases that could impact the contents of the article, e.g., the purity or stability of the drug product or maintaining a vacuum. Barrier properties can be targeted by using a single layer coating having low porosity and high surface energy (e.g., densely packed crystalline PTFE) or by applying multilayer films assembled with intermediate layer(s) to reduce permeability to oxygen and other gases (e.g., PVOH layer combined with crystalline PTFE top coating). Alternatively, inorganic nanoparticles can be included in an iCVD deposited coating.

Plastic tubes coated on the interior wall surface with the barrier film coating(s) of the present invention can maintain substantially better vacuum retention, draw volume and thermomechanical integrity retention than plastic tubes comprised of polymer compositions and blends thereof with a barrier film coating on the external wall surface of the tube. In addition, the resistance of the tube to impact is substantially much better than that of glass. Most notable is the clarity of the barrier film coating and its durability to substantially withstand resistance to impact and abrasion, such as during shipping and handling for syringes, use in automated machinery such as centrifuges for testing tubes and/or exposure to certain levels of radiation in the sterilization process.

As discussed above, in some non-limiting embodiments, the present invention encompasses articles of manufacture comprising a first component having a contact surface in frictional engagement with a contact surface of a second component. These articles can be used in any field in which components are in sliding engagement, for example medical articles, etc. As used herein, “medical article” means an article of manufacture or device that can be useful for medical treatment. Non-limiting examples of medical articles include articles selected from the group consisting of a syringe assembly, drug cartridge, needleless injector, liquid dispensing device, liquid metering device, sample collection tube or plate assembly, catheter, and vial. In some non-limiting embodiments, the medical article is a syringe assembly comprising a syringe chamber or barrel (for receiving water, saline or a medicament, for example) and a sealing member.

While not intending to be limited, the present invention now will be discussed with respect to first and second components of medical articles, using chambers (containers) and sealing members.

In some non-limiting embodiments, the chamber is selected from the group consisting of a syringe barrel, liquid container, and tube. The chamber can be formed from glass, metal, ceramic, plastic, rubber or combinations thereof. In some non-limiting embodiments, the chamber is prepared from Type I borosilicate glass. In some non-limiting embodiments, the chamber is prepared from one or more olefinic polymers, such as polyethylene, polypropylene, poly(1-butene), poly(2-methyl-1-pentene), and/or cyclic polyolefins. For example, the polyolefin can be a homopolymer or a copolymer of an aliphatic monoolefin, the aliphatic monoolefin preferably having about 2 to 6 carbon atoms, such as polypropylene. In some non-limiting embodiments, the polyolefin can be basically linear, but optionally may contain side chains such as are found, for instance, in conventional, low density polyethylene. In some non-limiting embodiments, the polyolefin is at least 50% isotactic. In other non-limiting embodiments, the polyolefin is at least about 90% isotactic in structure. In some non-limiting embodiments, syndiotactic polymers can be used. In some embodiments, cyclic polyolefins can be used. Non-limiting examples of suitable cyclic polyolefins include dicyclopentadiene (DCP), norbornene, tetracyclododecene (TCD), alternating, random or block ethylene/norbonanediyl units, or other polymeric type units such as are disclosed in U.S. Pat. Nos. 6,525,144, 6,511,756, 5,599,882, and 5,034,482 (each of Nippon Zeon), 7,037,993, 6,995,226, 6,908,970, 6,653,424 and 6,486,264 (each of Zeon Corp.), 7,026,401, and 6,951,898 (Ticona), 6,063,886 (Mitsui Chemicals), 5,866,662, 5,856,414, 5,623,039 and 5,610,253 (Hoechst), 5,854,349, and 5,650,471 (Mitsui Petrochemical and Hoechst) and as described in “Polycyclic olefins”, process Economics Program (July 1998) SRI Consulting, each of the foregoing references being incorporated by reference herein. Non-limiting examples of suitable cyclic polyolefins include Apel™ cyclic polyolefins available from Mitsui Petrochemical, Topas™ cyclic polyolefins available from Ticona Engineering Polymers, Zeonor™ or Zeonex™ cyclic polyolefins available from Zeon Corporation, and cyclic polyolefins available from Promerus LLC.

The polyolefin can contain a small amount, generally from about 0.1 to 10 percent, of an additional polymer incorporated into the composition by copolymerization with the appropriate monomer. Such copolymers may be added to the composition to enhance other characteristics of the final composition, and may be, for example, polyacrylate, polystyrene, and the like.

In some non-limiting embodiments, the chamber may be constructed of a polyolefin composition which includes a radiation stabilizing additive to impart radiation stability to the container, such as a mobilizing additive which contributes to the radiation stability of the container, such as for example those disclosed in U.S. Pat. Nos. 4,959,402 and 4,994,552, assigned to Becton, Dickinson and Company and both of which are incorporated herein by reference.

In some non-limiting embodiments, the chamber or container of the present invention is a blood collection device. The blood collection device can be either an evacuated blood collection tube or a non-evacuated blood collection tube. The blood collection tube can be made of polyethylene terephthalate, polypropylene, polycarbonate, polycycloolefin, polyethylene naphthalate or copolymers thereof.

The dimensions, e.g., inner and outer diameter, length, wall thickness, etc. of the chamber can be of any size desired. For example, for a one ml volume syringe barrel, the inner diameter of the barrel is about 0.25 inches (6.35 mm) and the length is about 2.0 inches (50.8 mm) For a plastic Sterifill 20 ml volume syringe barrel, the inner diameter of the barrel is about 0.75 inches (19.05 mm) and the length is about 3.75 inches (95.3 mm) Generally, the inner diameter can range from about 0.25 inches (6.35 mm) to about 10 inches (254 mm), or about 0.25 inches (6.35 mm) to about 5 inches (127 mm), or any value therebetween.

The other component of the medical article in contact with the chamber is the sealing member. The sealing member can be formed from any elastomeric or plastic material. Elastomers are used in many important and critical applications in medical devices and pharmaceutical packaging. As a class of materials, their unique characteristics, such as flexibility, resilience, extendability, and sealability, have proven particularly well suited for products such as catheters, syringe tips, drug vial articles, tubing, gloves, and hoses. Three primary synthetic thermoset elastomers typically are used in medical applications: polyisoprene rubber, silicone rubber, and butyl rubber. Of the three rubbers, butyl rubber has been the most common choice for articles due to its high cleanness and permeation resistance which enables the rubber to protect oxygen- and water-sensitive drugs.

Suitable butyl rubbers useful in the present invention include copolymers of isobutylene (about 97-98%) and isoprene (about 2-3%). The butyl rubber can be halogenated with chlorine or bromine. Suitable butyl rubber vulcanizates can provide good abrasion resistance, excellent impermeability to gases, a high dielectric constant, excellent resistance to aging and sunlight, and superior shock-absorbing and vibration-damping qualities to articles formed therefrom. Non-limiting examples of suitable rubber stoppers include those available from West Pharmaceuticals, American Gasket Rubber, Stelmi, and Helvoet Rubber & Plastic Technologies BV.

Other useful elastomeric copolymers include, without limitation, thermoplastic elastomers, thermoplastic vulcanizates, styrene copolymers such as styrene-butadiene (SBR or SBS) copolymers, styrene-ethylene/butylene-styrene (SEBS) copolymers, styrene-ethylene/propylene-styrene (SEPS) copolymers, styrene-isoprene (SIS) block polymers or styrene-isoprene/butadiene (SIBS), in which the content of styrene in the styrene block copolymer ranges from about 10% to about 70%, and preferably from about 20% to about 50%. Non-limiting examples of suitable styrene-butadiene stoppers are available from Firestone Polymers, Dow, Reichhold, Kokoku Rubber Inc., and Chemix Ltd. Other suitable thermoplastic elastomers are available from GLS, Tecknor Apex, AES, Mitsubishi and Solvay Engineered Polymers, for example. The elastomer composition can include, without limitation, antioxidants, UV resistance additives and/or inorganic reinforcing agents to preserve the stability of the elastomer composition.

In some embodiments, the sealing member can be a stopper, O-ring, V-ring, plunger tip, or piston, for example. Syringe plunger tips or pistons typically are made of a compressible, resilient material such as rubber, because of the rubber's ability to provide a seal between the plunger and interior housing of the syringe. Syringe plungers, like other equipment used in the care and treatment of patients, have to meet high performance standards, such as the ability to provide a tight seal between the plunger and the barrel of the syringe.

In some non-limiting embodiments, the average surface roughness (Rₐ) of the contact or outer surface of the chamber and/or sealing member ranges from about 10 nm to about 1700 nm, or about 10 nm to about 400 nm, or about 300 nm to about 1000 nm determined at about 20° C. to about 40° C., or about 25° C. The average surface roughness (Rₐ) correlates to the texture of the surface, and is quantified by the vertical deviations of a real surface from an idealized form of the surface. Rₐ is the arithmetic average of the absolute values of amplitude parameters based on the vertical deviations of a roughness profile from the mean line. The average surface roughness (Rₐ) can be determined using the formula:

$${R}_{a} = \frac{1}{n} {\sum }_{i = 1}^{n} \vert {y}_{i} \vert$$

wherein n is the number of data points, yᵢ is the vertical distance from the mean line to the i^{th} data point. This formula assumes that the mean line has been calculated from the raw data. The average surface roughness (Rₐ) can be determined using a non-contact optical interferometric profilometer such as Model No. Wyko NT1100, which is available from Veeco of Plainview, N.Y.

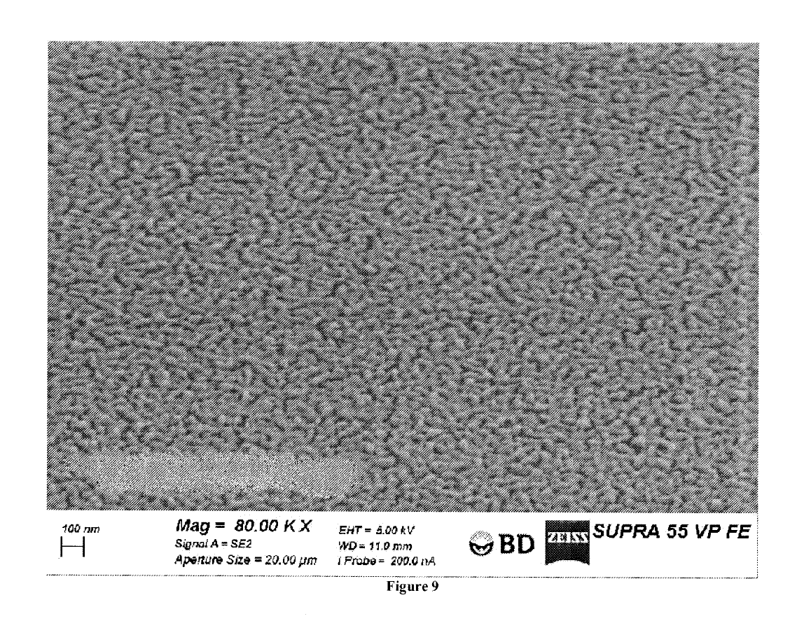

This surface roughness and coating morphological structure of the contact surface can provide cavities adjacent to the contact surface that permit gas(es) (such as air and/or nitrogen) and/or liquid(s) (such as silicone oil, water and/or fluorinated oils) to be trapped at the contact surface and present at the interface between contacting surfaces of adjacent components to facilitate sliding relative thereto, as shown in FIG. 9. In some non-limiting embodiments, the mean average diameter of the cavity openings ranges from about 0.5 nm to about 500 nm. The mean average diameter of the cavity openings can be determined by Fourier Transform image processing or SEM to measure each cavity opening over a predetermined surface area of the contact surface (such as 1 mm×1 mm), and calculating the mean average of the values measured. The diameter for each cavity is measured as the largest diameter across each cavity.

This surface roughness of the contact surface can be provided by the coating layer itself, or the coating layer can be a sublayer that contributes to the desired surface roughness of the contact surface.

The coating layer is applied to at least a portion of the of the chamber and/or sealing member. In some embodiments, the chamber is coated with the coating described below and the sealing member is uncoated or coated with a polydimethylsiloxane coating. In other embodiments, the sealing member is coated with the coating described below and the chamber is uncoated or coated with a polydimethylsiloxane coating. In other embodiments, both the chamber and sealing member are coated with coatings as described below. Methods for coating the surface(s) are discussed in detail below.

The chamber and/or sealing member can be coated with a coating layer prepared from a composition comprising one or more polymers selected from the group consisting of poly(tetrafluoroethylene) (“PTFE”), ultra high molecular weight poly(ethylene) (“UHMWPE”), poly(vinylidene fluoride) (“PVF”), poly(amide), poly(propylene), poly(p-phenylene vinylene) (“PPV”), poly(p-phenylene sulfide) (“PPS”) and combinations thereof. In some non-limiting embodiments, the coating layer comprises, consists essentially of, or consists of poly(tetrafluoroethylene). Optionally, organosilicon can be included in the coating layer. The thickness of the coating layer can range from about 10 nm to about 20 μm, or about 500 nm to about 1000 nm, or about 1000 nm to about 20 μm.

In some non-limiting embodiments, the coating layer comprises crystalline domains, wherein the mass of the crystalline domains comprises at least about 20% of the total mass of the coating layer, or at least about 30%, or at least about 40%, or at least about 50%, or at least about 60%, or at least about 70% of the total mass of the coating layer. In some non-limiting embodiments, the coating layer comprises about 20% to about 99% mass of crystalline domains based upon the total mass of the coating layer. The mass of the crystalline domains can be determined by XRD using a Bruker GADDS microdiffractometer 500 mm pinhole collimator, Cu—Kα line 1.54 angstroms wavelength, scattering angle collection 10-70 Two-theta degrees in a manner well known to those skilled in the art. For a PTFE coating, the peak occurs at about 18 degrees for the Two-theta angle. Alternatively, the percent crystallinity can be determined by Differential Scanning calorimetry (DSC) at 2° C./min in a manner well known to those skilled in the art. In some non-limiting embodiments, the coating comprises lamellar (semi)crystalline polymers, such as (semi)crystalline poly(tetrafluoroethylene) (“PTFE”), ultra high molecular weight poly(ethylene) (“UHMWPE”), poly(vinylidene fluoride) (“PVF”), poly(amide), poly(propylene), poly(p-phenylene vinylene) (“PPV”), poly(p-phenylene sulfide) (“PPS”) and combinations thereof.

In some non-limiting embodiments, the coating layer can be prepared by hot filament chemical vapor deposition, plasma-enhanced chemical vapor deposition, glow discharge, melt emulsion casting, spinning, or electrochemical or solution polymerization crystallization, or physical vapor deposition. Such deposition methods are well known to those skilled in the art. Suitable conditions for such depositions can be determined through routine experimentation to provide a coating layer having the desired roughness and/or crystallinity. For example, the coating layer can be deposited by PECVD in a manner described in A. Millela et al., “Deposition Mechanism of Nanostructured Thin Films from Tetrafluoroethylene Glow Discharges”, Pure App. Chem., Vol. 77, No. 2, pp. 399-414 (2005), incorporated by reference herein. The coating layer can be deposited by solution polymerization in a manner described in D. Lin et al., “On the Structure of Porous Poly(vinylidene fluoride) Membrane Prepared by Phase Inversion from Water-NMP-PVDF System”, Tamkang J. Sci. and Egr., Vol. 5, No. 2 (2002) at pages 95-98. The coating layer can be deposited by electrochemical solution polymerization in a manner described in N. Sonoyama et al., “Electrochemical Conversion of CFC-12 to Tetrafluoroethylene: Electrochemical Formation of Difluorocarbene”, Electrochimica Acta 47, pp. 3847-3851 (2002).

In some non-limiting embodiments, the coating layer is prepared by hot filament chemical vapor deposition. For example, the coating layer can be deposited by hot filament vapor deposition methods such as are described in U.S. Pat. Nos. 5,888,591, 6,153,269, 6,156,435, and 6,887,578, each incorporated by reference herein.

A suitable PTFE coating can be deposited by hot filament chemical vapor deposition of at least one halocarbon or fluorocarbon monomer selected from the group consisting of hexafluoropropylene oxide, tetrafluoroethylene, hexafluorocyclopropane, octafluorocyclobutane, perfluorooctanesulfonyl fluoride, octafluoropropane, trifluoromethane, difluoromethane, difluorodichloromethane, difluorodibromomethane, difluorobromomethane, difluorochloromethane, trifluorochloromethane, tetrafluorocyclopropane, tetrachlorodifluorocyclopropane, trichlorotrifluoroethane, dichlorotetrafluorocyclopropane and mixtures thereof. The term “fluorocarbon” as used herein means a halocarbon compound in which fluorine replaces some or all hydrogen atoms.

Optionally, organosilicon monomers, azurine monomers, thiirane monomers, unsaturated olefinic monomers and mixtures thereof can be included in amounts up to about 90 weight percent. The term “organosilicon” as used herein means a compound containing at least one Si—C bond. Non-limiting examples of suitable organosilicon monomers include those selected from the group consisting of hexamethylcyclotrisiloxane, octamethylcyclotetrasiloxane, 1,3,5-trivinyl-1,3,5-trimethylcyclotrisiloxane, 1,3,5,7-tetravinyl-1,3,5,7-tetramethylcyclotrisiloxane, 3-(N-allylamino)propyltrimethoxysilane, allyldichlorosilane, allyldimethoxysilane, allyldimethylsilane, allyltrichlorosilane, allyltrimethoxysilane, allyltrimethylsilane, bis(dimethylamino)vinylmethylsilane, para-(t-butyldimethylsiloxy)styrene, decamethylcyclopentasiloxane, diethylsilane, dimethylethoxysilane, dimethylsilane, divinyldimethylsilane, divinyltetramethyldisilane, 1,3-divinyltetramethyldisiloxane, ethyltrimethoxysilane, hexamethyldisiloxane, 1,1,3,3,5,5-hexamethyltrisiloxane, hexavinyldisiloxane, methyltriethoxysilane, methyltrimethoxysilane, methylsilane, tetraethoxysilane, tetraethylcyclotetrasiloxane, tetraethylsilane, tetramethoxysilane, 1,1,3,3-tetramethyldisiloxane, tetramethylsilane, tetravinylsilane, trimethylsilane, vinyldimethylsilane, vinylmethylbis(trimethylsiloxy)-silane 3-vinylheptamethyltrisiloxane, vinylmethyldiethoxysilane, vinyloxytrimethylsilane, vinylpentamethyldisiloxane, vinyltetramethyldisiloxane, vinyltrimethoxysilane, vinyltrimethylsilane, and mixtures thereof.

Non-limiting examples of suitable unsaturated olefin monomers include those selected from the group consisting of dicyclopentadiene (DCP), dipentadiene, norbornene, cyclopentadiene, methyltetracyclododecene (MTD), tetracyclododecene, and mixtures thereof.

In some non-limiting embodiments, the deposition processes enable tailoring of the chemical composition of deposited films to produce fluorocarbon polymer thin films having stoichiometry and materials properties similar to that of bulk PTFE. One useful monomer is hexafluoropropylene oxide (C₃F₆O or HFPO). HFPO is characterized by a highly-strained epoxide ring that enables easy ring-opening reactions with nucleophiles. It has been found that films deposited using HFPO under HFCVD conditions result in polymer films having a high CF₂ fraction and little or no oxygen incorporation.

In some non-limiting embodiments, the processes of the present invention contemplate use of any feed gas that provides a monomer which can be pyrolyzed to provide difluorocarbene species (CF₂) for producing a fluorocarbon polymer film having a high fraction of CF₂ groups and a low degree of polymer crosslinking. For example, the HFPO monomer described above is understood to decompose under pyrolysis to form a fluorinated ketone and the desired difluorocarbene. The fluorinated ketone is relatively stable, compared with the difluorocarbene. This is understood to lead to a high CF₂ content in a film as polymerization occurs at the film deposition surface. Oxygen present in the monomer is tied up in the relatively unreactive ketone decomposition byproduct, whereby little oxygen is incorporated into the film.

Considering the selection of gas monomer in general, the ratio of CFₓ/F in the gas can effect the competing deposition and etching reactions that occur during a deposition process; a higher ratio can correspond to enhancement of deposition and suppression of etching reactions. This ratio can be increased by including in a feed gas composition a fluorine scavenger, e.g., hydrogen, a hydrocarbon, or an unsaturated compound or monomer. In general, the addition of hydrogen or C₂F₄ to a fluorocarbon feed gas can result in decreasing atomic F concentration relative to CFₓ concentration. This decreased atomic F concentration can result in an increased deposition rate. Additionally, the inclusion of hydrogen in the feed gas can alter the gap-filling capabilities of the deposited film due to its reduction in ion bombardment. Furthermore, hydrogen can be included in a feed gas to provide an in situ mechanism for passivating dangling bonds on the surface of a structure being processed. For example, hydrogen can passivate amorphous silicon dangling bonds. In some non-limiting embodiments, use of less reactive, but not interfering, radicals than the difluorocarbene or the use of an on-off deposition scheme where the input of the gases is alternated can be used.

The selection of feed gas constituents also preferably should take into consideration any trace impurities that could be incorporated into a film deposited from the feed gas. For example, HFPO as a feed gas monomer can result in incorporation of trace amount of oxygen in a deposited film. Thus, if trace oxygen is not acceptable for a deposited film, a feed gas monomer other than HFPO is preferable. Other process parameters should likewise preferably be considered in selecting a feed gas monomer, as will be recognized by those skilled in the art.

In some non-limiting embodiments, the monomer gas can comprise azurines or thiirane oxides, for example 3-methyl-2H-azirine-2-methyl, 3-amino-2H-azirine-2-methyl, 2-hydroxy-2H-azirine, 3-phenyl-2H-azirine, 2,3-Diarylthiirene 1-oxide, 2,3-di-t-butylthiirrene 1-oxide, and 2,3-Dimethylthiirene.

Chemically different monomer gases can be used sequentially to deposit multiple layers upon the substrate. Alternatively, if a blend of reactive gases is used, the volume ratio of the blend can be adjusted during deposition to form multiple layers or a gradient layer. Non-limiting examples of multilayer deposition materials include deposition of a mixture of polyethylene and siloxane as a first layer and PTFE as a second layer, or PTFE as a first layer and poly acrylic acid as a second layer.

The temperature of the pyrolyzing or hot filament surface should be sufficient to pyrolyze or combust at least a portion of the monomer gas and form one or more reactive moieties. For example, the temperature of the pyrolyzing surface can be about 300° K to about 773° K, or greater than about 500° K. One skilled in the art would understand that the monomer gas need not directly contact the pyrolyzing surface, but can be at least partially pyrolyzed when in proximity to the heat generated by the pyrolyzing surface. Also, one skilled in the art would understand that, depending upon the monomer gas(es) selected, the pyrolysis temperature and duration of exposure may vary. One skilled in the art would understand that the monomer gas pyrolysis and subsequent polymerization could be influenced (catalyzed or stereodirected) by the type of metal present in the surface of the hot filament or other surface close to the pyrolysis area.

The term “chemical vapor deposition” as used herein means a process which transforms gaseous molecules or radicals into solid material in the form of thin film or powder on the surface of a substrate. In the thermal or hot filament chemical vapor deposition (thermal-CVD) process, substantially no ion bombardment occurs, because no substantial electric field is generated in the deposition chamber to attract the charged ions to the film as it is deposited. Notably, and in contrast to films deposited by PECVD, films deposited via hot-filament CVD (HFCVD) have well-defined compositions. For example, PECVD-deposited fluorocarbon films comprise a variety of CF groups (e.g., CF₃, tertiary C, and C—F, in addition to CF₂), while HFCVD-deposited fluorocarbon films consist almost entirely of CF₂, along with a small amount of CF₃ moieties. Further, the initiating and terminating groups in HFCVD are well-defined; whereas the precursors in PECVD processes undergo much greater fragmentation (these films have Si—F bonds, for instance, that result from total fragmentation of the fluorocarbon precursors). A consequence of the nature of the HFCVD process is that only the most thermally stable groups (e.g., CF₂ and siloxane rings) appear in the film, resulting in more thermally stable films. Photo-initiated CVD (ph-CVD), or photolysis, has the specificity of HFCVD with the advantage of minimal or no collateral thermal heat being added to the system allowing deposition under cooler conditions, can also be used. This ph-CVD can be used in conjunction with the presence of an activating surface as mentioned previously to increase the efficiency of the process.

One of the most important specific chemical differences between hot-filament CVD and plasma-enhanced CVD is the occurrence of ion-bombardment and ultraviolet-irradiation in the latter technique. Due to this difference, HFCVD films do not contain defects seen in PECVD films. For example, HFCVD films do not have dangling bonds, which are always produced in PECVD processes. Dangling bonds are unpaired electrons left behind in the film. If such bonds are present, the film will undergo reactions with components of the ambient atmosphere (such as water, for instance, resulting in a large number of hydroxyl groups). Therefore, PECVD films are more susceptible to atmospheric ageing, and degradation of their optical, electrical and chemical properties. Moreover, films produced by HFCVD processes are less dense than those produced by plasma-enhanced CVD processes. Due to the differences between the nucleation and growth mechanisms the two processes, it is possible to make porous films using HFCVD, but not using PECVD.

The use of an initiator in HFCVD allows films to be deposited at significantly higher rates and provides greater control over chemical composition and morphology. This was demonstrated by Pryce Lewis et al. for fluorocarbon films deposited from hexafluoropropylene oxide (HFPO) using perfluorooctane sulfonyl fluoride (PFOSF) as an initiator. Pryce Lewis, H. G.; Caulfield, J. A.; Gleason, K. K. Langmuir 2001, 17, 7652. In the mechanism proposed for film growth, the generation of free radicals from the pyrolysis of PFOSF is the initiation step. The fluorocarbon radical subsequently combines with the propagating species, difluorocarbene (CF₂), which is generated by the pyrolysis of HFPO. The use of PFOSF resulted in higher deposition rates, more efficient utilization of HFPO, and endcapping by CF₃ groups.

In some non-limiting embodiments, the coating layer has a first region deposited from a first reactive gas having a chemical functionality that is reactive with a first surface functionality present in a first domain of the interior wall surface prior to coating and a second region deposited from a second reactive gas having a chemical functionality that is reactive with a second surface functionality present in a second domain of the interior wall surface prior to coating. Such chemical functionalities can include carbon-carbon unsaturation, nitrile, imido, amido or halo functionality, for example. For example, the interior wall surface of the chamber can comprise regions or domains of different chemical functionality or properties, for example a first domain can have carbon to carbon double bond (unsaturated) chemical functionality and a second domain can have ester chemical functionality. Reactive gases having different reactive functionalities can be selectively deposited onto these first and second domains deposited from reactive gases having respective functionalities that are reactive with the surface functionality present in each domain. For example, if the first domain has C═C chemical functionality, a reactive gas such as dicyclopentene having diene chemical functionality can be deposited to react with the C═C chemical functionality of the first domain. Similarly, if the second domain has ketone chemical functionality, a reactive gas such as oxygen having can be concurrently or sequentially deposited to react with the ketone chemical functionality of the second domain. Thus, the final coating can have regions different chemical identities having different physical properties, for example hydrophilic and hydrophobic regions.

For example, the interior wall surface of the chamber can be formed from a mixture of cyclic polyolefin and SEBS, providing domains of SEBS rich in unsaturated bonds. The unsaturated bonds are more likely to react with the difluorocarbene radical than saturated carbon domains. Thus the regions with a heavier concentration of PTFE can provide regions of greater lubricity, and other regions can be tailored using other reactive gases to provide other physical characteristics, such as hydrophilicity or hydrophobicity.

The coating layer is formed by exposing the surface to the first and second reactive gases, either sequentially or simultaneously, and exposing the coated surface to an energy source to facilitate formation of the coating layer. In some non-limiting embodiments, the coating layer is applied by chemical vapor deposition, such as plasma CVD or hot filament CVD. In other non-limiting embodiments, the coating layer is exposed to oxidative treatment to facilitate formation of the coating layer, or a combination thereof.

In some non-limiting embodiments, the pyrolyzing surface can be a hot-filament. The hot-filament or other heated surface is preferably provided in a position relative to the input monomer gas flow such that the input monomer gas flows in the vicinity of the heated structure; whereby the gas is pyrolyzed to produce reactive deposition species. The hot filament can be heated by, e.g., resistive heating. In this case, a dc voltage source is provided to apply the heating voltage to the filament, consisting of, e.g., a Ni/Cr wire. The hot filament wire can have a diameter of about 0.3 to about 0.5 mm, for example, and a length to provide the appropriate ohmic resistance to adjust the temperature of the process as well as effective area to be coated.

Among the different CVD techniques available, hot-filament CVD (HFCVD, also known as pyrolytic or hot-wire CVD) is unique in several respects. In HFCVD, a precursor gas is thermally decomposed by a resistively heated filament. The resulting pyrolysis products adsorb onto a substrate maintained at around room temperature and react to form a film. HFCVD does not require the generation of plasma, thereby avoiding defects in the growing film produced by UV irradiation and ion bombardment. In addition, films produced by HFCVD have a better-defined chemical structure because there are fewer reaction pathways than in the less selective plasma-enhanced CVD method. HFCVD provides films with a substantially lower density of dangling bonds, i.e., unpaired electrons. Further, HFCVD has been shown to produce films that have a low degree of crosslinking. HFCVD has been used to deposit fluorocarbon films that are spectroscopically similar to poly(tetrafluoroethylene) (PTFE). Limb, S. J., Lau, K. K. S., Edell, D. J., Gleason, E. F., Gleason, K. K. Plasmas and Polymers 1999, 4, 21.

Thermal excitation mechanisms other than a hot-filament are equally suitable for the thermal-CVD process. Indeed, it is preferable that the selected thermal mechanism, together with the gas delivery system, provide both uniform gas input and uniform pyrolysis of the gas. Hot windows, electrodes, or other surfaces can alternatively be employed in pyrolysis configurations aimed at producing uniform gas pyrolysis. Other direct heating techniques, e.g., laser heating techniques, can also be employed, as can be employed in general a wide range of other pyrolysis mechanisms.

In some non-limiting embodiments using HFCVD, the temperature of the substrate upon which the coating is to be deposited, e.g., the chamber or sealing member, is maintained at a temperature which is less than the temperature of the pyrolyzing surface or reactive gas to facilitate deposition and polymerization of the reactive moiety on at least a portion of the interior wall surface of the chamber or container. The temperature of the interior wall surface of the chamber is maintained at a temperature lower than the pyrolysis temperature of the pyrolyzing surface or reactive gas. Specifically, the temperature of the interior wall surface of the chamber is preferably maintained low enough to favor polymerization under the partial pressure of a given reactive species employed in the deposition process. It is also preferable that the partial pressure of the reactive species be kept to a low level that prevents homogeneous gas-phase reactions, which could cause particle production in the gaseous environment rather than on the object surface to be coated.

In some non-limiting embodiments, the temperature of the interior wall surface of the chamber depends of the specific material being coated and the cross section for establishing covalent bonding between the radical species and the surface, e.g., for cyclic polyolefin resin the temperature must be lower than 140° C. The temperature of the interior wall surface is less than the temperature of the pyrolyzing surface or reactive gas, for example at least about 20° C. or more less, and in some embodiments is held at a temperature of between about −40° C. and about +200° C. (233° K to 473° K) during the deposition; or about 20° C. to about 50° C. (293° K to 323° K).

The temperature that is maintained during film deposition can be an important factor for determining the deposition rate, the stability of the radical species and the ultimate thermal stability of a film produced by the deposition process. Films deposited at relatively higher structural temperatures may in some applications be relatively more resistant to heating. The deposition time will depend on the flow rate, activation efficiency and targeted thickness of the coating. Typical deposition times can range from seconds to hours. Very fast deposition times are desirable to implement the on-off scheme deposition scheme mentioned above. The thickness of the film generally can range from about 1 nm to about 100 microns, for example.

In some non-limiting embodiments, the contact surface is further treated with a surface treatment (oxidative, noble gas or other), heat treatment, and/or irradiation with an isotope, electron beam, or ultraviolet radiation. This additional treatment can be carried out prior to, simultaneously with, or after the pyrolysis treatment. This treatment can promote adhesion (covalent or non-covalent bonding) or surface property modifications.

The plasma treatment may be carried out in any common vacuum or atmospheric plasma generation equipment. Any suitable ionizing plasma may be used, as, for example, a plasma generated by a glow discharge or a corona discharge. The plasma may be generated from a variety of gases or mixtures thereof. Gases frequently used include air, hydrogen, helium, ammonia, nitrogen, oxygen, neon, argon, krypton, and xenon. Any gas pressure may be used, for example, atmospheric pressure or 5 mm of Hg or below, such as about 0.1 to about 1.0 mm of Hg. In some embodiments such as atmospheric oxidative methods, the ionizing plasma can be introduced directly from a small port at the opening in the chamber. In other embodiments, such as vacuum based equipment, the plasma can be excited around the coated chamber and allowed to diffuse into the chamber features. Alternatively, the plasma may be excited within the interior of the open chamber by properly controlling electrode position. After oxidative treatment, the treated chamber can be subjected to heat treatment or irradiation with an isotope (such as gamma radiation), electron beam, or ultraviolet radiation. Alternatively, the treated chamber can be heat treated via oven or radio frequency (RF). In the case of oven crosslinking, temperatures can range from about 120° to about 140° C. and residence time in the oven is generally about 30 to about 40 seconds, depending on the precise formulation. If RF techniques are used, the coil should conduct enough heat to obtain a substrate surface temperature of about 150° to about 200° C. At these temperatures, only about 2 to about 4 seconds are required for cure.

In some non-limiting embodiments, the coating is at least partially crosslinked by irradiation with an isotope, electron beam, or ultraviolet radiation. This technique has the advantage of sterilizing as well, which is useful in medical applications. Radiation sterilization in the form of ionizing radiation commonly is used in hospitals for medical devices such as catheters, surgical items, and critical care tools. Gamma irradiation exerts a microbicidal effect by oxidizing biological tissue, and thus provides a simple, rapid and efficacious method of sterilization. Gamma rays are used either from a cobalt-60 (⁶⁰Co) isotope source or from a machine-generated accelerated electron source. Sufficient exposures are achieved when the materials to be sterilized are moved around an exposed ⁶⁰Co source for a defined period of time. The most commonly used dose for sterilizing medical articles is about 5 to about 100 kGy, for example, 5-50 kGy.

In some non-limiting embodiments, the coating composition further comprises at least one inorganic material. In some non-limiting embodiments, the inorganic material particles are formed from solid lubricant materials. As used herein, the term “solid lubricant” means any solid used between two surfaces to provide protection from damage during relative movement and/or to reduce friction and wear. As used herein, “inorganic solid lubricant” means that the solid lubricants have a characteristic crystalline habit which causes them to shear into thin, flat plates which readily slide over one another and thus produce an antifriction lubricating effect. See R. Lewis, Sr., Hawley's Condensed Chemical Dictionary, (12th Ed. 1993) at page 712, incorporated by reference herein.

In some non-limiting embodiments, the particles have a lamellar structure. Particles having a lamellar structure are composed of sheets or plates of atoms in hexagonal array, with strong bonding within the sheet and weak van der Waals bonding between sheets, providing low shear strength between sheets. A non-limiting example of a lamellar structure is a hexagonal crystal structure. Inorganic solid particles having a lamellar fullerene (i.e., buckyball) structure can also be useful in the present invention.

In some non-limiting embodiments, the contact surface of the chamber or sealing member is subjected to at least one treatment selected from the group consisting of oxidative treatment, heat treatment, and irradiation with an isotope, electron beam, or ultraviolet radiation prior to, simultaneously with, or after the pyrolysis treatment, as discussed in detail above.

In some non-limiting embodiments, the coating can be polymerized using a photolysis energy source having a predetermined wavelength (or range of wavelengths). In some non-limiting embodiments, the photolysis energy source is ultraviolet radiation having a predetermined wavelength within the ultraviolet range. In some non-limiting embodiments, the photolysis energy source is gamma radiation having a predetermined wavelength within the gamma range. In some non-limiting embodiments, the photolysis energy source is obtained from a laser source. The photolysis can be performed from outside the container (for example shining the light beam through the container walls in the case of a transparent container) or inside the container (for example with a collinear annular beam directed from the open end to the second end of the container). The source would be a tunable (selective) light source consisting for example of a tunable laser (using dye, or n-harmonic generation crystals) or a white light source coupled to a filter, for example a laser-driven-light source (such as LDLS EQ-99 from Energetiq Technology, Inc. of MA). In the case of photolysis, the filament or other heating source can be used to enhance the catalysis but is not required not for performing the pyrolysis of the monomer gas.

In some non-limiting embodiments, the coated articles are subjected to a sterilization treatment. Commonly used sterilization techniques used for medical devices include autoclaving, ethylene oxide (EtO) or gamma irradiation, as well as more recently introduced systems that involve low-temperature gas plasma and vapor phase sterilants.

The mating contact surface of the other component (not coated according to the present invention discussed above) can be coated with a conventional siloxane or other oil coating as described above. The surface lubricant can be conventional silicone oil (organopolysiloxane) of viscosity about 100 to 1,000,000; 100 to 60,000; or preferably about 1,000 to 12,500 cSt, evaluated using a Brookfield DV II+ viscometer. The surface lubricating layer may be applied by any conventional method, such as spraying or dipping the stopper into a solution, about 4% by weight, of the surface lubricant in a solvent such as chloroform, dichloromethane or preferably a chlorofluorocarbon, such as FREON™ TF. The surface lubricant may optionally be lightly crosslinked by oxidative treatment and/or radiation.

In some non-limiting embodiments, the present invention provides a method for lubricating the interface between an inner surface of a chamber and an exterior surface of a sealing member of a medical article, comprising: applying a coating onto the interior surface of the chamber and/or the exterior surface of the sealing member to provide a contact surface thereon having an average surface roughness (Rₐ) ranging from about 10 nm to about 1700 nm.

In some non-limiting embodiments, the present invention provides a method for reducing breakloose force between an inner surface of a chamber and an exterior surface of a sealing member of a medical article, comprising: applying a coating onto the interior surface of the chamber and/or the exterior surface of the sealing member to provide a contact surface thereon having an average surface roughness (Rₐ) ranging from about 10 nm to about 1700 nm.

In some non-limiting embodiments, the present invention provides a method for reducing sustaining force between an inner surface of a chamber and an exterior surface of a sealing member of a medical article, comprising: applying a coating onto the interior surface of the chamber and/or the exterior surface of the sealing member to provide a contact surface thereon having an average surface roughness (Rₐ) ranging from about 10 nm to about 1700 nm.

In some non-limiting embodiments, the present invention provides a method for reducing sticktion between an inner surface of a chamber and an exterior surface of a sealing member of a medical article, comprising: applying a coating onto the interior surface of the chamber and/or the exterior surface of the sealing member to provide a contact surface thereon having an average surface roughness (Rₐ) ranging from about 10 nm to about 1700 nm.

In some non-limiting embodiments, the present invention provides a method for lubricating the interface between an inner surface of a chamber and an exterior surface of a sealing member of a medical article, comprising: applying a coating onto the interior surface of the chamber and/or the exterior surface of the sealing member, the coating layer comprising crystalline domains, wherein the mass of the crystalline domains comprises at least about 20% of the total mass of the coating layer.

In some non-limiting embodiments, the present invention provides a method for reducing breakloose force between an inner surface of a chamber and an exterior surface of a sealing member of a medical article, comprising: applying a coating onto the interior surface of the chamber and/or the exterior surface of the sealing member, the coating layer comprising crystalline domains, wherein the mass of the crystalline domains comprises at least about 20% of the total mass of the coating layer.

In some non-limiting embodiments, the present invention provides a method for reducing sustaining force between an inner surface of a chamber and an exterior surface of a sealing member of a medical article, comprising: applying a coating onto the interior surface of the chamber and/or the exterior surface of the sealing member, the coating layer comprising crystalline domains, wherein the mass of the crystalline domains comprises at least about 20% of the total mass of the coating layer.

In some non-limiting embodiments, the present invention provides a method for reducing sticktion between an inner surface of a chamber and an exterior surface of a sealing member of a medical article, comprising: applying a coating onto the interior surface of the chamber and/or the exterior surface of the sealing member, the coating layer comprising crystalline domains, wherein the mass of the crystalline domains comprises at least about 20% of the total mass of the coating layer.

The present invention is more particularly described in the following examples, which are intended to be illustrative only, as numerous modifications and variations therein will be apparent to those skilled in the art.

### EXAMPLE

Cyclic polyolefin (dicyclopentadiene-tricyclodecane) substrate plaques, Helvoet FM457 butyl rubber stoppers (W4023 1 ml stopper), silicon wafers or glass substrates were coated with poly(tetrafluoroethylene) coatings according to the present invention by HFCVD using a Ni/Cr wire in a manner described in U.S. Pat. No. 6,887,578. The monomer gas was hexafluoropropylene oxide. The filament was maintained at a temperature of about 673° K and the substrate surface was maintained at a temperature below 50° C. and a monomer flow rate of about 25 sccm. The thickness of each coating layer is specified below.

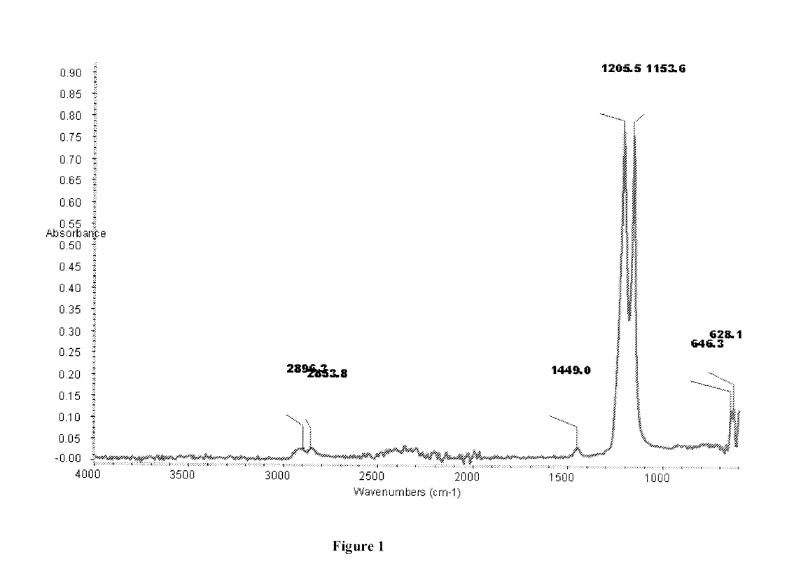

FIG. 1 is an FTIR analysis of a portion of a 500 nm thick semi-crystalline PTFE sample on a cyclic polyolefin substrate showing the presence of the two CF₂ bands at approximately 1200 and 1150 cm⁻¹, indicating that the coating is substantially poly(tetrafluoroethylene).

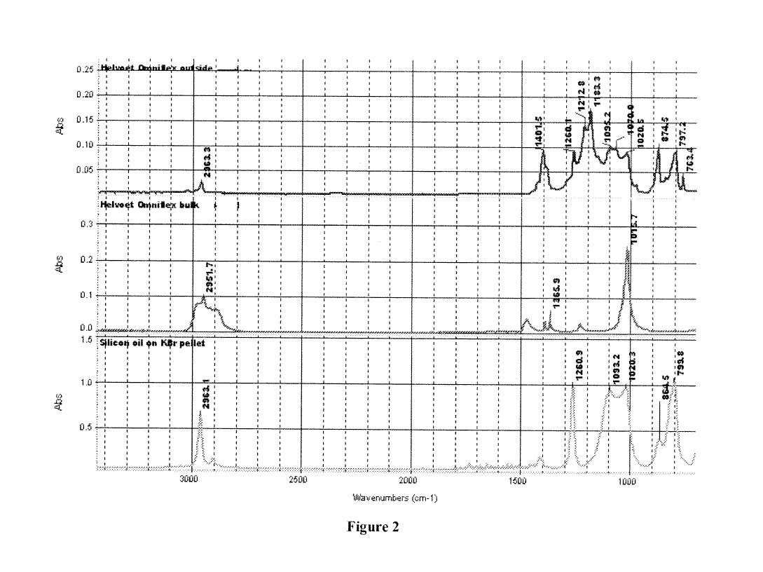

For comparison, FIG. 2 is an FTIR of a non-crystalline Omniflex® fluoro-coated rubber stopper, showing the FTIR signal from the rubber (bulk) and the signal from the surface. The appearance of the two CF₂ peaks at 1213 and 1183 cm⁻¹ is clearly distinguishable from the contributions of bulk rubber (mainly by the silicate peak at 1183 cm⁻¹) and lubrication fluid of the stopper (by the silicone oil peaks 1260, 1095, 1020 and 797 cm⁻¹). The FTIR analyses were conducted using a ThermoNicolet Magna 760 spectrometer, 8 scans and 4000-600 cm⁻¹ range.

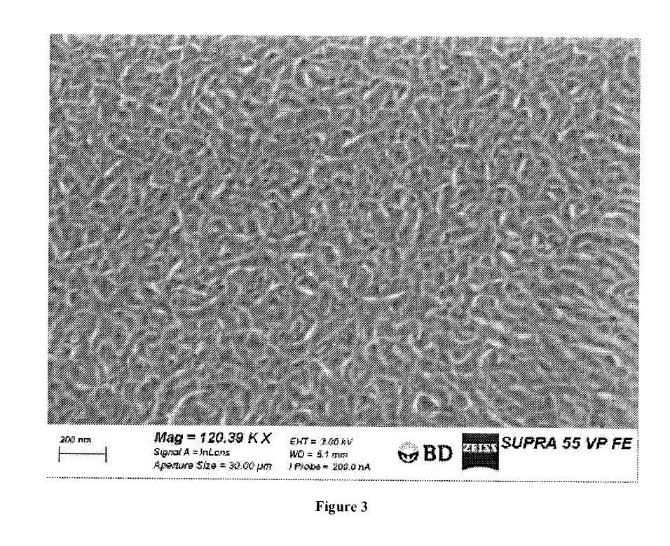

Another portion of the 500 nm thick semi-crystalline PTFE-coated cyclic polyolefin sample of FIG. 1 was analyzed to determine the mass of crystalline PTFE based upon total mass of the coating layer using a Zeiss Supra V55 emission filament electron microscope (SEM), frame average of 4, 5 KV electron beam acceleration. FIG. 3 shows a porous (filamentary) structure due to the growth of the crystals along the long axis of the crystal.

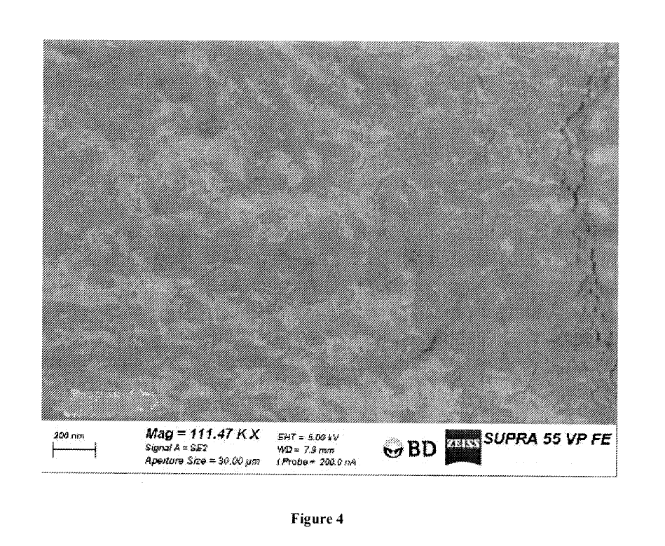

In contrast, SEM analysis of a portion of the Omniflex® fluoro-coated rubber stopper shown in FIG. 4) reveals a very different, amorphous (non-crystalline) morphology.

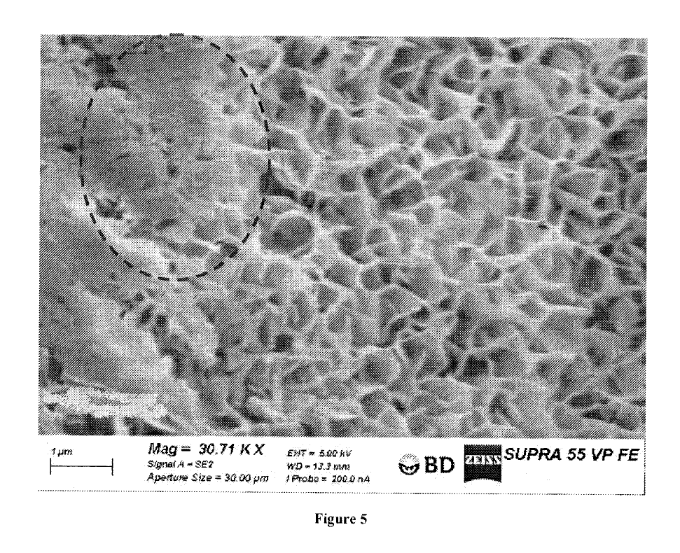

FIG. 5 is an SEM analysis of a portion of a 12 μm thick semi-crystalline PTFE coating on a butyl rubber stopper according to the present invention, showing a honeycomb-like structure. The PTFE coating had a crystallinity of about 70% based upon total mass.

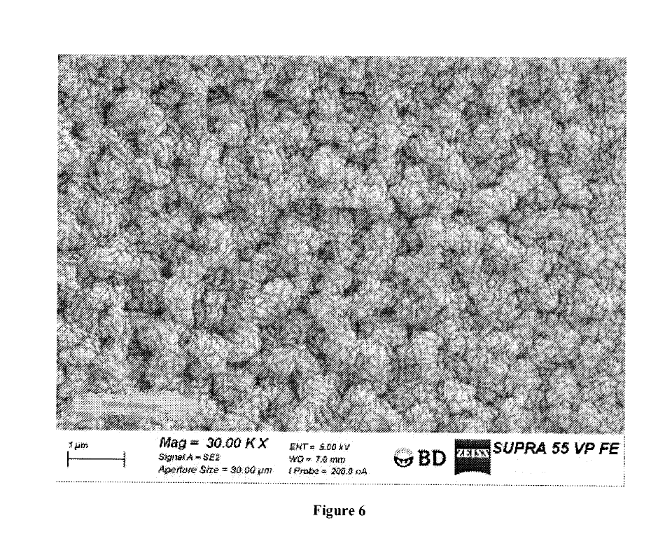

FIG. 6 is an SEM analysis of a portion of an 8 μm thick semi-crystalline PTFE coating on a silicon wafer substrate according to the present invention, showing another type of semi-crystalline morphology.

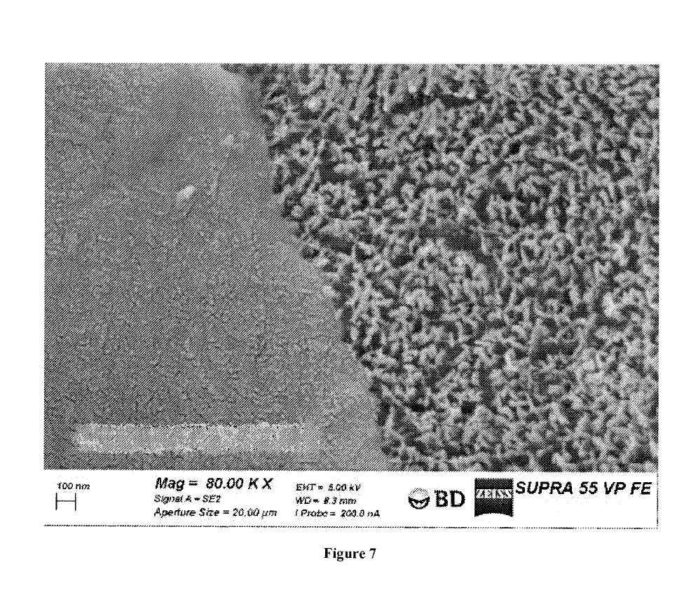

FIG. 7 is an SEM analysis of a portion of a 500 nm thick semi-crystalline PTFE coating on a butyl rubber stopper according to the present invention, showing two different crystalline morphologies growing simultaneously on the substrate. The phase on the left side of the photograph is very densely packed. The phase on the right side is less densely packed.

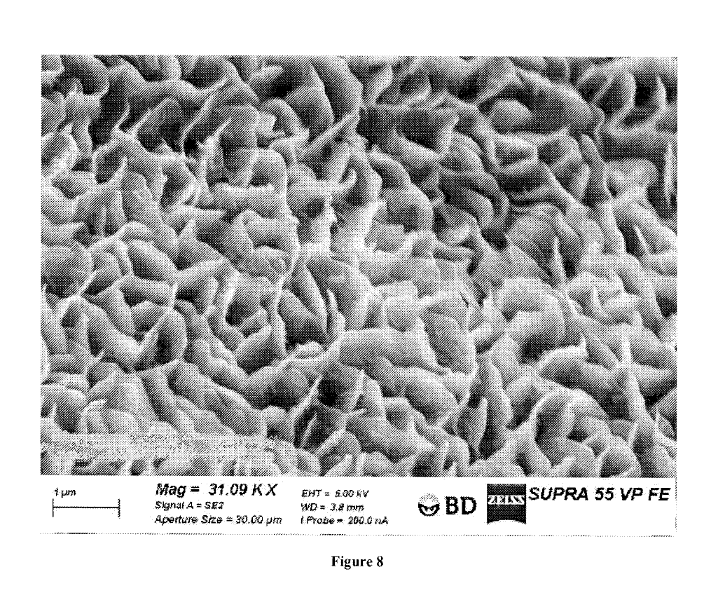

FIG. 8 is an SEM analysis of a portion of an 8 μm thick semi-crystalline PTFE coating on a butyl rubber plate according to the present invention. The size of the micro/nano cavities and/or pores creates a very high energy surface which impedes the penetration of water or polar fluids into the structure. If, on the other hand, a non-polar fluid is embedded in this structure, the forces required to displace this fluid are very high because of the capillarity effect.

FIG. 9 is an SEM analysis of a 25 nm thick semi-crystalline polytetrafluoroethylene-coated glass substrate, according to the present invention.

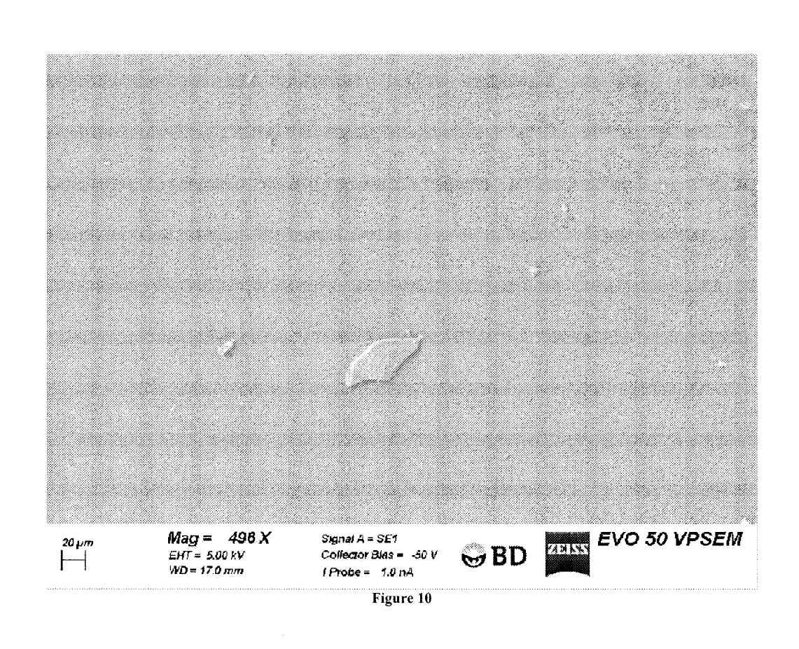

FIG. 10 is an SEM analysis of a 1.4 μm thick semi-crystalline polytetrafluoroethylene-coated fragment of a glass syringe barrel, according to the present invention.

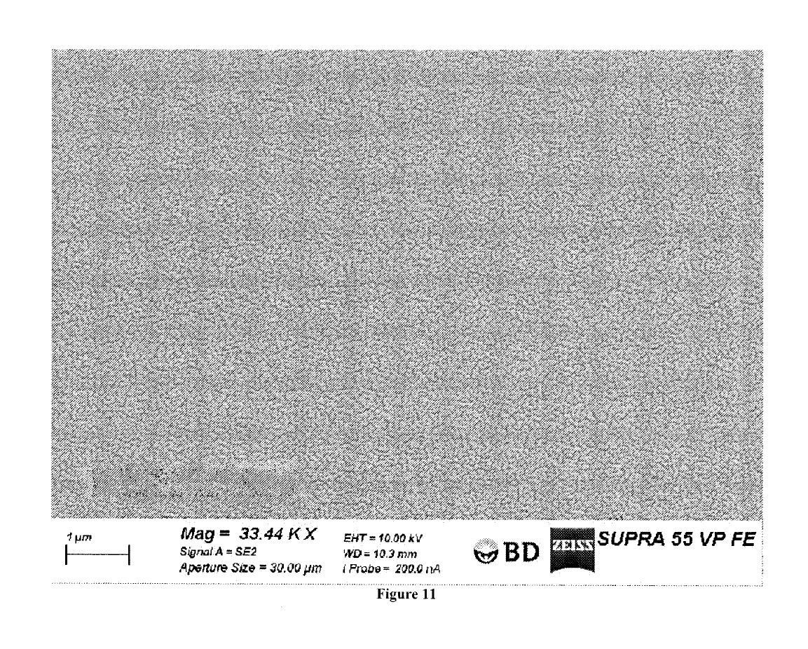

FIG. 11 is an SEM analysis of a 1.4 μm thick semi-crystalline polytetrafluoroethylene-coated fragment of a cyclic polyolefin syringe barrel, according to the present invention.

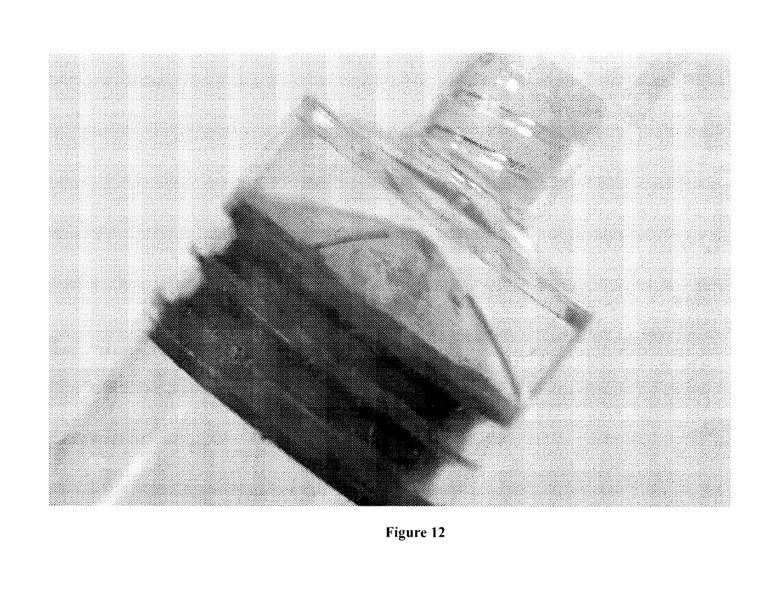

FIG. 12 is a photograph of a syringe assembly showing a butyl rubber sealing member coated with a 4 μm thick PTFE semi-crystalline coating (70% crystalline in mass) in a syringe barrel, according to the present invention. The photograph shows the light reflection from a film of air trapped in the surface cavities of the coating. This sample had been subjected to one week of stability testing at 40° C. for infusion pump actuation force according to ISO 7886-2 Annex A, as described below, prior to photography.

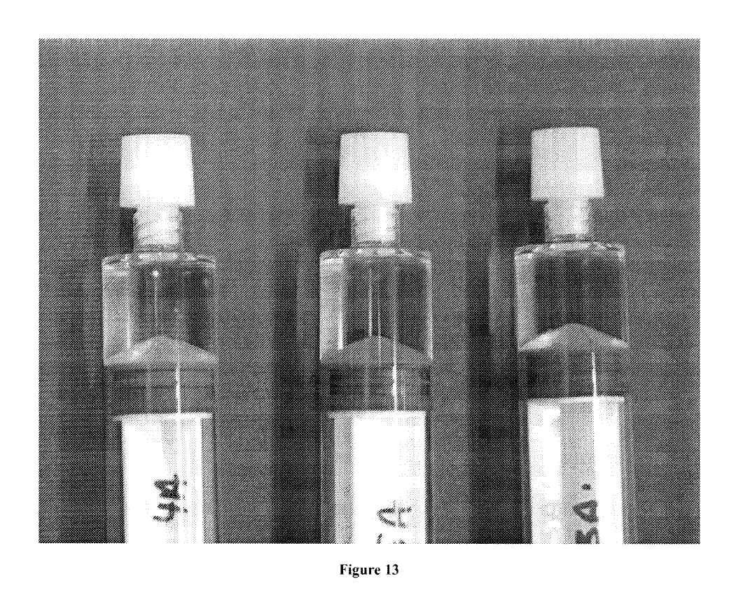

FIG. 13 is a photograph of semi-crystalline polytetrafluoroethylene-coated 20 ml butyl rubber stoppers within respective syringe barrels (left side (4A) and right side (3A)), according to the present invention and a conventional 20 ml siliconized stopper available from Helvoet or Becton Dickinson (center). The photograph shows the light reflection from a film of air trapped in the surface cavities of the coatings of stoppers 4A and 3A. The silicon coated stopper in the center shows no light reflection. Each of these samples had been subjected to 4 months of stability testing at −40° C. for infusion pump actuation force according to ISO 7886-2 Annex A, as described below, prior to photography.

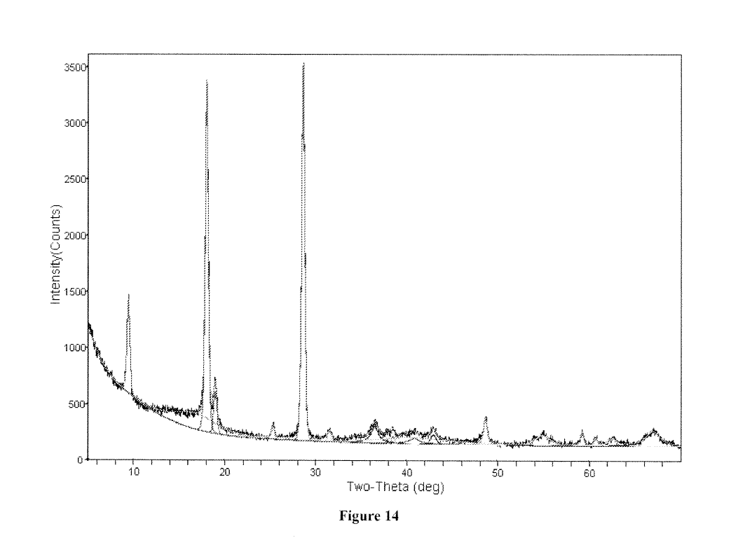

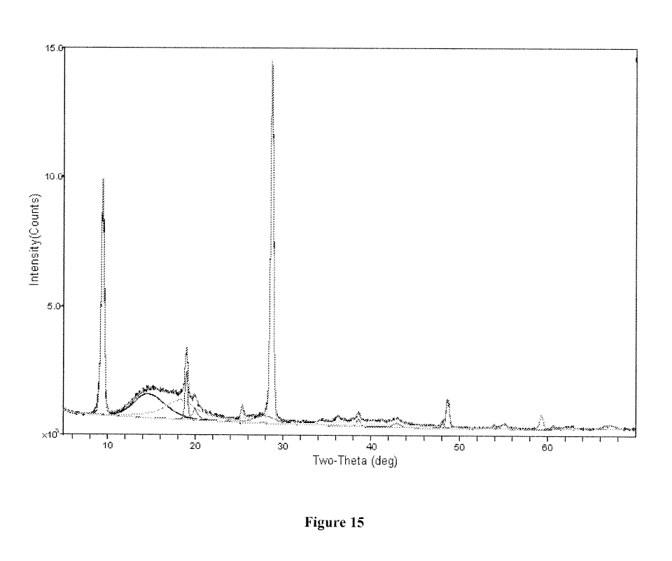

FIG. 14 is an X-ray diffraction (XRD) analysis of a 20 μm thick semi-crystalline polytetrafluoroethylene-coated butyl rubber substrate, according to the present invention. FIG. 15 is an XRD analysis of a 25 μm thick Omniflex® fluoro-coated rubber stopper. The XRD analysis was conducted using a Bruker GADDS microdiffractometer 500 mm pinhole collimator, Cu—Kα line 1.54 angstroms wavelength, scattering angle collection 10-70 2*theta degrees). FIG. 14 shows crystallinity at 18 degrees for the 2 theta angle PTFE crystalline. This peak is absent for the Omniflex® fluoro-coating (FIG. 15). Peaks observed in Omniflex® fluoro-coating at 18.4 degrees 2 theta are believed to correspond to crystallinity from fillers in the bulk rubber. The comparison between FIGS. 14 and 15 shows that in this case of two butyl rubbers they use similar filler, possibly rutile titanium dioxide.

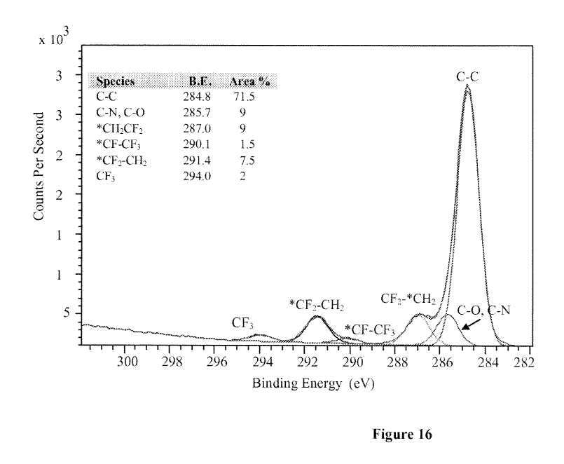

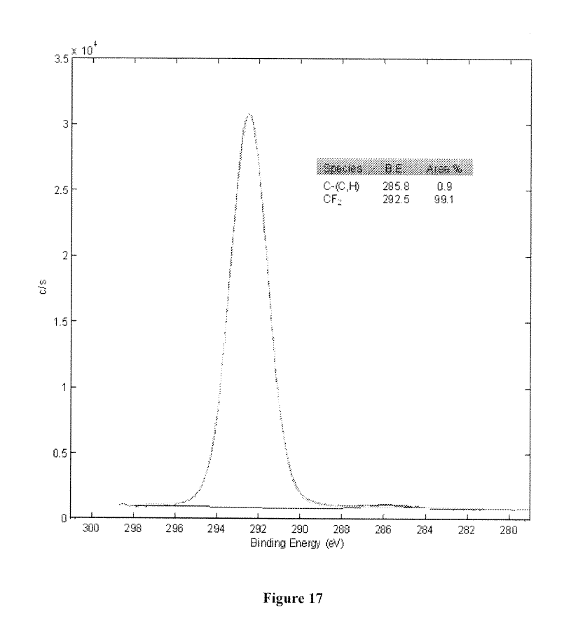

FIG. 16 is an X-ray photoelectron spectroscopy (XPS) analysis of an Omniflex® 25 μm thick fluoro-coated 20 ml butyl rubber stopper. FIG. 17 is an X-ray photoelectron spectroscopy (XPS) analysis of a semi-crystalline polytetrafluoroethylene-coated (8 μm thickness) 20 ml butyl rubber stopper, according to the present invention.

Analytical Parameters

| | |
|---|---|
| Instrument | PHI 5701 LSci |
| X-ray source | Monochromated Alk_{α} 1486.6 eV |
| Acceptance Angle | ±7° |
| Take-off angle | 20° & 50° |
| Analysis area | 2.0 mm × 0.8 mm |
| Charge Correction | C1s 284.8 eV |

The atomic concentrations and carbon chemical bonding predicted from the analysis of the C1s, O1s, Si 2s, and N 1s bands are shown in Tables 1 and 2.

TABLE 1
Atomic Concentrations (in %)^{a}

| Sample | C | F | O | Si | N |
|---|---|---|---|---|---|
| Omniflex ® coating | 47.7 | 10.6 | 22.7 | 18.9 | 0.1 |
| PTFE coating | 30.9 | 68.8 | 0.3 | — | — |

^{a}Normalized to 100% of the elements detected. XPS does not detect H or He.
^{b} A dash line “—” indicates the element is not detected.

TABLE 2
Carbon Chemical State (in % of Total C^{a}) from
the analysis of C1s band

| Sample | C—C | C—(O,N) | CF₂—*CH₂ | CF—CF₃ | *CF₂—CH₂ | CF₂ | CF₃ |
|---|---|---|---|---|---|---|---|
| Omniflex ® | 71.5 | 9 | 9 | 1.5 | 7.5 | — | 2 |
| coating | | | | | | | |
| PTFE | 1 | — | — | — | — | 99 | — |
| coating | | | | | | | |

^{a}Values in this table are percentages of the total atomic concentration of the corresponding element shown in Table 1.

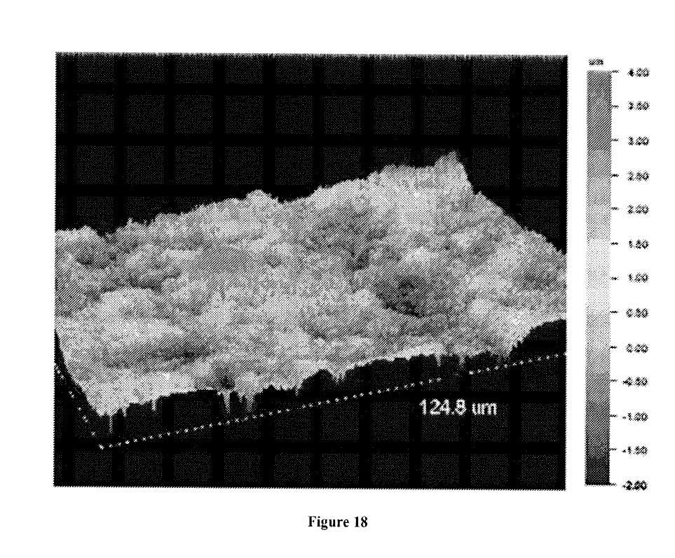

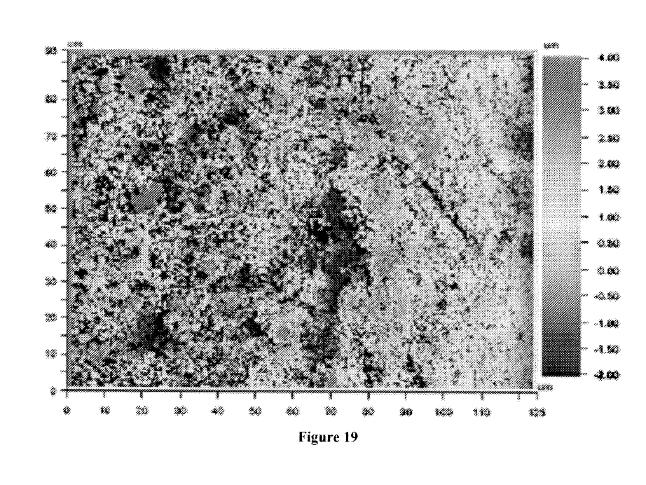

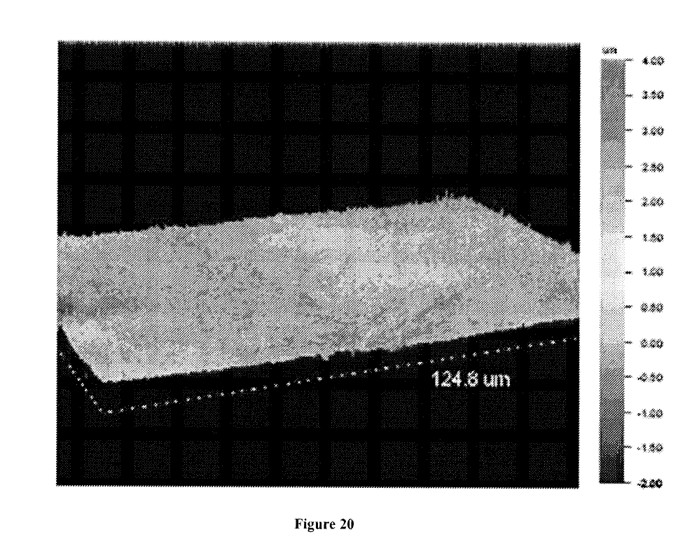

The average surface roughness (Ra) was determined using a Veeco Model No. Wyko NT1100 non-contact optical interferometric profilometer. The measurements were taken over an optical field of about 124.8 μm×52.9 μm. FIG. 18 is an optical profilometry analysis of a semi-crystalline polytetrafluoroethylene-coated (8 μm thickness) butyl rubber stopper, according to the present invention. As shown in FIG. 18, the average surface roughness (Ra) for the sample was 841 nm±30 nm. FIG. 19 is an optical profilometry analysis of a semi-crystalline polytetrafluoroethylene-coated (8 μm thickness) butyl rubber stopper, according to the present invention. As shown in FIG. 19, the average surface roughness (Ra) for the sample was 1510 nm. FIG. 20 is an optical profilometry analysis of an Omniflex® fluoro-coated (25 μm thickness) rubber stopper. As shown in FIG. 20, the average surface roughness (Ra) for the sample was 181 nm±20 nm.

Breakout forces, breakloose forces, and sustaining forces may be conveniently measured on a universal mechanical tester or on a testing machine of the type having a constant rate of cross-head movement, as described in detail below. Selected syringe assemblies were evaluated for breakloose force according to ISO 7886-1 Annex G. The breakloose (actuation) and sustaining force (in kilograms) of each sample syringe was determined by an Instron Series 5500 at a displacement rate of 380 mm/min according to ISO 7886. The breakloose force is visually determined as the highest peak of the curve or point of where the slope of the curve changes on the graph. The sustaining force is the average force for the stopper to move an additional 25-30 mm for a 1 ml barrel or an additional 115-120 mm for a 20 ml barrel after breakloose. The breakloose and sustaining values reported in Table 3 below are the results of two samples each. The gliding performance can be summarized by the absolute value of the force required to displace the stopper from initial position (indicated by “ACT” on the figure) and the maximum value of the force needed to sustain its displacement (indicated by “GF” on the figure). Lower values are representative of higher performance.

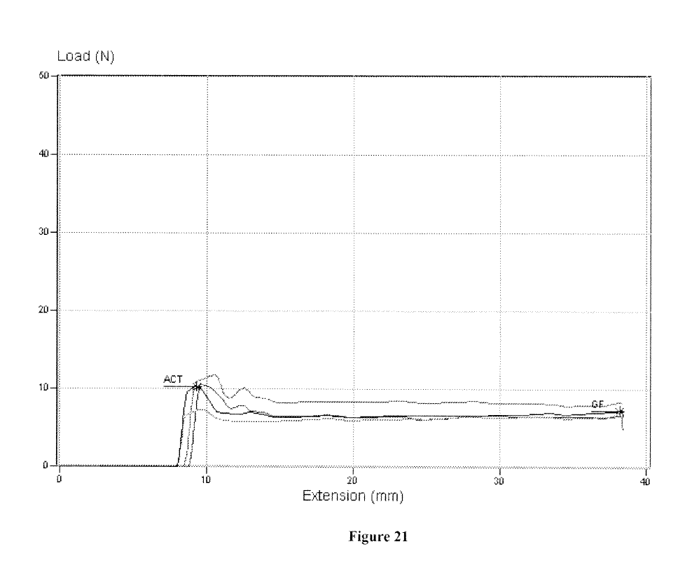

FIG. 21 is a graph of actuation and gliding force between a semi-crystalline polytetrafluoroethylene-coated (8 μm thickness) butyl rubber stopper and a non-lubricated glass barrel, according to the present invention. The four lines correspond to four replicate samples for the same coated substrate.

Table 3 presents values of activation and gliding forces for different coatings on 1 ml glass syringes with a 27 gauge needle attached, filled with water for injection (WFI) compression testing performed at a constant speed of 380 mm/min. Table 3 shows that the semi-crystalline PTFE coated rubber stopper having an average surface roughness Rₐ of 830 nm provided comparable activation force and lower maximum gliding force compared to a thicker Omniflex® non-crystalline PTFE coated stopper when used with an uncoated glass barrel. When used with an uncoated cyclic polyolefin barrel, the semi-crystalline PTFE coated rubber stopper having an average surface roughness Rₐ of 830 nm provided lower activation and lower maximum gliding force compared to a thicker Omniflex® non-crystalline PTFE coated stopper.

TABLE 3

| | Stopper | Crystal- | Roughness | Activation | Max gliding |
|---|---|---|---|---|---|
| Substrate | Coating | linity % | Rₐ (nm) | force (N) | force (N) |
| Bare | 8 μm PTFE | 71% | 830 | 9.9 | 7.8 |
| glass | | | | | |
| Bare | 25 μm | 0% | 181 | 7.6 | 44.0 |
| glass | Omniflex ® | | | | |
| Cyclic | 8 μm PTFE | 71% | 830 | 14.3 | 34.5 |
| polyolefin | | | | | |
| Cyclic | 25 μm | 0% | 181 | >50 | >50 |
| polyolefin | Omniflex ® | | | | |

XRD measured crystallinity of the samples varied because of the diffusive X-ray scattering contributions from the bulk rubber as the beam sampled different amounts of bulk rubber.

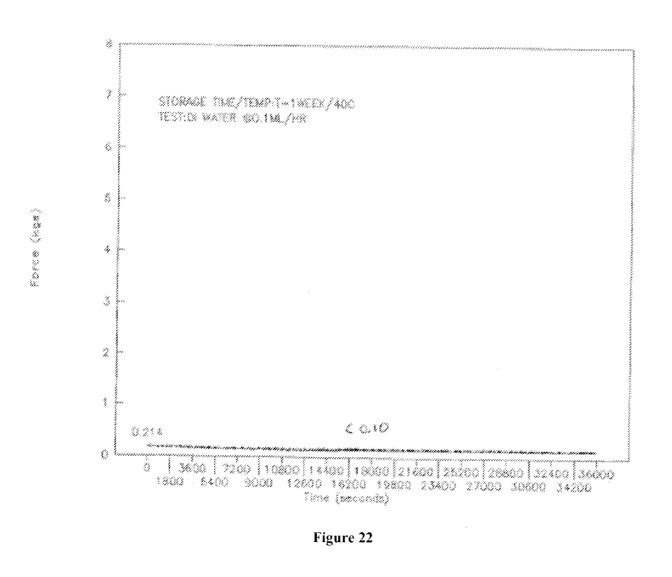

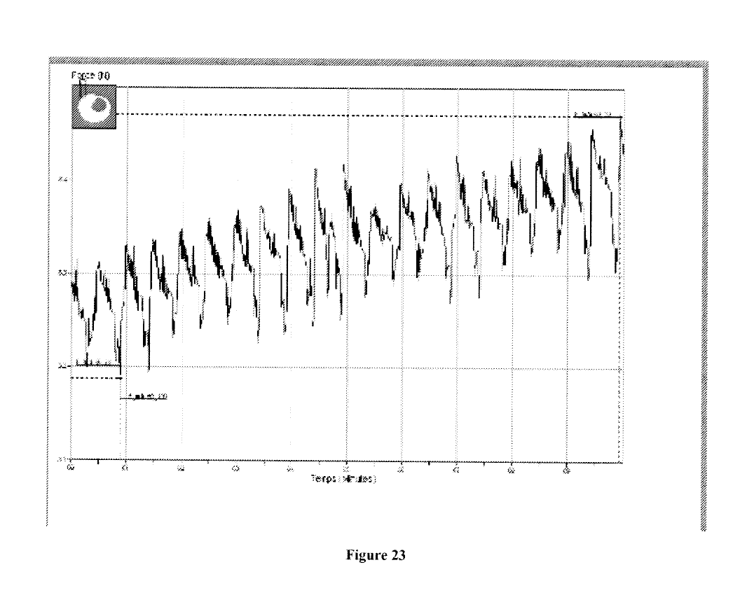

Selected syringe assemblies were evaluated for infusion pump actuation force according to ISO 7886-2 Annex A. A Becton Dickinson Program 2 syringe pump was used for testing at a flow rate of 0.1 ml/hr and displacement of 0.03 mm/min. Force was measured using a force transducer placed between the syringe plunger rod and the displacement arm of the pump. A chart of force over time for each syringe was generated, as shown in FIGS. 22 and 23. FIG. 22 is a graph of infusion pump actuation force test results (kg_{f}) for a semi-crystalline polytetrafluoroethylene-coated (4 μm thickness) rubber stopper and a conventional silicone oil lubricated 20 ml barrel syringe assembly at a feed rate of 0.1 ml/hr, according to the present invention. FIG. 23 is a graph of infusion pump actuation force test results (N) for an Omniflex® fluoro-coated (25 μm thickness) rubber stopper and a 20 ml conventional silicone oil lubricated barrel syringe assembly at a feed rate of 0.1 ml/hr.

A visual determination of sticktion or no sticktion can be made by viewing each chart for the smoothness of the curve. A smooth curve indicated no sticktion and an irregularly-shaped curve (for example with discernable peaks) indicated sticktion. The sample tested in FIG. 22 (semi-crystalline polytetrafluoroethylene-coated (4 μm thickness) rubber stopper according to the present invention) shows less sticktion than the Omniflex® non-crystalline coated stopper of FIG. 23.

The present invention has been described with reference to specific details of particular embodiments thereof. It is not intended that such details be regarded as limitations upon the scope of the invention except insofar as and to the extent that they are included in the accompanying claims.

What is claimed is:

1. An article of manufacture comprising a first component having a contact surface in frictional engagement with a contact surface of a second component, wherein at least one of the first component and the second component comprises a substrate having at least one coating layer on at least a portion of a surface of the substrate, wherein the contact surface of the first and/or second component has an average surface roughness (Rₐ) ranging from about 10 nm to about 1700 nm.

2. The article of manufacture according to claim 1, wherein the article is selected from the group consisting of a syringe assembly, drug cartridge, needleless injector, liquid dispensing device, liquid metering device, sample collection tube or plate assembly, catheter, and vial.

3. The article of manufacture according to claim 1, wherein the first component is selected from the group consisting of a syringe barrel, liquid container, and tube.

4. The article of manufacture according to claim 1, wherein the first component is formed from glass, metal, ceramic, plastic, or combinations thereof.

5. The article of manufacture according to claim 4, wherein the first component is prepared from an olefinic polymer selected from the group consisting of polyethylene, polypropylene, poly(1-butene), poly(2-methyl-1-pentene), cyclic polyolefins, and mixtures thereof.

6. The article of manufacture according to claim 1, wherein the second component is a sealing member.

7. The article of manufacture according to claim 6, wherein the sealing member is selected from the group consisting of a stopper, O-ring, V-ring, plunger tip, and piston.

8. The article of manufacture according to claim 6, wherein the sealing member is formed from rubber.

9. The article of manufacture according to claim 1, wherein the coating layer comprises the contact surface.

10. The article of manufacture according to claim 1, wherein the coating layer comprises a polymer selected from the group consisting of poly(tetrafluoroethylene), ultra high molecular weight poly(ethylene), poly(vinylidene fluoride), poly(amide), poly(propylene), poly(p-phenylene vinylene), poly(p-phenylene sulfide) and combinations thereof.

11. The article of manufacture according to claim 1, wherein the coating layer comprises poly(tetrafluoroethylene).

12. The article of manufacture according to claim 1, wherein the coating layer comprises crystalline domains, wherein the mass of the crystalline domains comprises at least about 20% of the total mass of the coating layer.

13. The article of manufacture according to claim 1, wherein the coating layer is prepared by hot filament chemical vapor deposition, plasma-enhanced chemical vapor deposition, glow discharge, melt emulsion casting, spinning, or electrochemical or solution polymerization or physical vapor deposition.

14. The article of manufacture according to claim 13, wherein the coating layer is prepared by hot filament chemical vapor deposition.

15. The article of manufacture according to claim 14, wherein the coating layer is prepared by hot filament chemical vapor deposition of at least one halocarbon monomer selected from the group consisting of hexafluoropropylene oxide, tetrafluoroethylene, hexafluorocyclopropane, octafluorocyclobutane, perfluorooctanesulfonyl fluoride, octafluoropropane, trifluoromethane, difluoromethane, difluorodichloromethane, difluorodibromomethane, difluorobromomethane, difluorochloromethane, trifluorochloromethane, tetrafluorocyclopropane, tetrachlorodifluorocyclopropane, trichlorotrifluoroethane, dichlorotetrafluorocyclopropane and mixtures thereof.

16. The article of manufacture according to claim 1, wherein the coating composition further comprises at least one inorganic material selected from the group consisting of graphite, talc, mica, and combinations thereof.

17. The article of manufacture according to claim 1, wherein average surface roughness (Rₐ) ranges from about 10 nm to about 400 nm.

18. A medical article comprising a chamber, the chamber comprising a substrate having at least one coating layer on at least a portion of a surface of the substrate, wherein an outer surface of the chamber has an average surface roughness (Rₐ) ranging from about 10 nm to about 1700 nm.

19. The medical article according to claim 18, wherein the coating layer comprises the outer surface of the chamber.

20. The medical article according to claim 18, wherein the coating layer comprises crystalline domains, wherein the mass of the crystalline domains comprises at least about 20% of the total mass of the coating layer.

21. A chamber for a medical article, the chamber having a contact surface adapted to sealingly engage a contact surface of a sealing member for a medical article, wherein the chamber comprises a substrate having at least one coating layer on at least a portion of a surface of the substrate, wherein the contact surface of the chamber has an average surface roughness (Rₐ) ranging from about 10 nm to about 1700 nm.

22. The chamber according to claim 21, wherein the coating layer comprises the contact surface of the chamber.

23. The chamber according to claim 21, wherein the coating layer comprises crystalline domains, wherein the mass of the crystalline domains comprises at least about 20% of the total mass of the coating layer.

24. A sealing member for a medical article, the sealing member having a contact surface in sliding engagement with a contact surface of a chamber of a medical article and adapted to sealingly engage the contact surface of the chamber, wherein the sealing member comprises a substrate having at least one coating layer on at least a portion of a surface of the substrate, wherein the contact surface of the sealing member has an average surface roughness (Rₐ) ranging from about 10 nm to about 1700 nm.

25. The sealing member according to claim 24, wherein the coating layer comprises the contact surface of the sealing member.

26. The sealing member according to claim 24, wherein the coating layer comprises crystalline domains, wherein the mass of the crystalline domains comprises at least about 20% of the total mass of the coating layer.
